(19) United States
(12) Patent Application Publication
(10) Pub. No.: US 2025/0134897 A1
(43) Pub. Date: May 1, 2025

(54) PREPARATION OF ISOTOPICALLY LABELED KETOGLUTARATES AND METHODS OF HYPERPOLARIZATION THROUGH SIGNAL AMPLIFICATION BY REVERSIBLE EXCHANGE (SABRE)

(71) Applicant: The United States of America,as represented by the Secretary,Department of Health and Human Services, Bethesda, MD (US)

(72) Inventors: Rolf E. SWENSON, Silver Spring, MD (US); Jessica H. ETTEDGUI-BENJAMINI, North Bethesda, MD (US); Venkatareddy SABBASANI, Monrovia, MD (US); Deepak SAIL, Potomac, MD (US); Kazutoshi YAMAMOTO, North Potomac, MD (US); Murali K. CHERUKURI, Potomac, MD (US)

(73) Assignee: The United States of America,as represented by the Secretary,Department of Health and Human Services, Bethesda, MD (US)

(21) Appl. No.: 18/833,627

(22) PCT Filed: Jan. 26, 2023
(86) PCT No.: PCT/US2023/011640

§ 371 Date: Jul. 26, 2024

Related U.S. Application Data
(60) Provisional application No. 63/303,190, filed on Jan. 26, 2022.

(51) Int. Cl. A61K 31/5375; A61K 31/194; C07C 51/41; C07C 59/347; C07D 295/185

(52) U.S. Cl. CPC A61K 31/5375; A61K 31/194; C07C 51/412; C07C 59/347; C07D 295/185

(57) ABSTRACT

Disclosed are compounds of Formula (I), pharmaceutically acceptable salts thereof, and methods of preparation thereof,

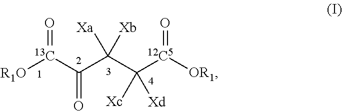

wherein R₁, Xa, Xb, Xc, and Xd are as described in the specification. Further disclosed is an isotopic mixture of a compound of Formula (I), as well as a pharmaceutical composition containing a hyperpolarized compound of Formula (I). Also disclosed is a method of diagnosing or monitoring a patient suffering from cancer, the method including administering a pharmaceutical composition comprising an effective amount of a hyperpolarized ketoglutarate compound or a pharmaceutically acceptable salt thereof, and measuring the hyperpolarization of a compound of interest in the patient. Further disclosed is a method of preparing hyperpolarized ketoglutarate compounds for use in the above methods.

### CROSS-REFERENCE TO PRIOR APPLICATIONS

This application claims benefit to U.S. Provisional Patent Application No. 63/303,190, filed Jan. 26, 2022, which is hereby incorporated by reference in its entirety.

### FEDERALLY SPONSORED RESEARCH OR DEVELOPMENT

This invention was made with Government support. The Government has certain rights in this invention.

### BACKGROUND OF THE INVENTION

Alphα-ketoglutarate (α-KG) is a key metabolite and signaling molecule in cancer cells, but the low permeability of α-KG limits the study of α-KG mediated effects in vivo. Cell-permeable monoester and diester α-KG derivatives have been synthesized for use in vivo, but many of these derivatives are not compatible for use in hyperpolarized carbon-13 nuclear magnetic resonance imaging (HP-¹³C-MRI). Certain isotopically labeled α-ketoglutarate compounds as well as their use as a hyperpolarized imaging, therapeutic, or diagnostic agents, and methods for their preparation have been disclosed. See, e.g., WO 2021/146572A1; AbuSalim et al., “Simple Esterification of [1-¹³C]-Alpha-Ketoglutarate Enhances Membrane Permeability and Allows for Noninvasive Tracing of Glutamate and Glutamine Production,” ACS Chem. Biol. 2021, 16, 2144-2150 (Sep. 23, 2021).

However, there still exists an unmet need for isotopically labeled α-KG compounds having an improved performance in HP-¹³C-MRI, for example, increased T1 relaxivity of the carbonyl group, as well as for a method of preparing such compounds, a method of preparing hyperpolarized isotopically labeled α-KG compounds, and a method of detecting and/or monitoring patients for various diseases by the use of these compounds.

### BRIEF SUMMARY OF THE INVENTION

The present invention provides isotopically labeled α-KG compounds having an improved performance in HP-¹³C-MRI, for example, an increased T1 relaxivity, i.e., the time required for the z-component of M to reach (1-1/e) or about 63% of its maximum value (Mo). More particularly, the present invention provides a compound of Formula (I):

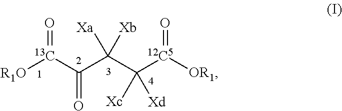

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

The present invention also provides a composition comprising an isotopic mixture of a compound of Formula (I):

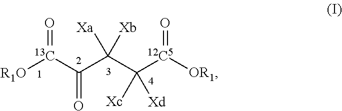

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, and wherein at least 25% of a sum total deuterium and hydrogen atoms defined by Xa+Xb+Xc+Xd are deuterium,
or a pharmaceutically acceptable salt thereof.

The present invention further provides a hyperpolarized compound of Formula (I), a hyperpolarized composition comprising an isotopic mixture of a compound of Formula (I), a pharmaceutical composition comprising an effective amount of a hyperpolarized compound of Formula I, and a pharmaceutical composition comprising a hyperpolarized isotopic mixture of a compound of Formula (I), or a pharmaceutically acceptable salt thereof, and a pharmaceutically acceptable carrier.

The present invention further provides a method of diagnosing or monitoring a patient having or suspected to have a cancer, the method comprising administering the hyperpolarized ketoglutarate compound, for example, a hyperpolarized compound of Formula (I), a hyperpolarized isotopic mixture of a compound of Formula (I), or a hyperpolarized compound of Formula (IV):

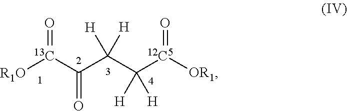

or a pharmaceutical composition comprising a hyperpolarized compound of Formula (I), a hyperpolarized isotopic mixture of a compound of Formula (I), or a hyperpolarized compound of Formula (IV), and diagnosing or monitoring the patient by hyperpolarized ¹³C-MRI.

The present invention also provides a method of preparing the compounds of Formula (I), as well as methods of preparing hyperpolarized variants and isotopic mixtures thereof.

### BRIEF DESCRIPTION OF THE DRAWINGS

FIG. 1 depicts synthetic schemes for preparing mono-3-deuterated ketoglutarates in accordance with aspects of the invention.

FIG. 2 depicts the NMR results obtained on compounds in an aspect of the invention. The top scan depicts a single-scan hyperpolarized (TIP)¹³C of sodium [1-¹³C, 5-¹²C] ketoglutarate. The bottom scan depicts a single-scan thermally polarized ¹³C signal from 4M sodium [1-¹³C]acetate.

FIGS. 3A-3H provide the results of the SABRE-SHEATH hyperpolarization process for [1-¹³C, 5-¹²C; d₄] α-ketoglutarate. FIG. 3A is a schematic showing the SABRE-SHEATH hyperpolarization process and relevant polarization transfer path of [1-¹³C, 5-¹²C; d₄] α-ketoglutarate. FIG. 3B provides the ¹³C NMR spectrum of hyperpolarized [1-¹³C, 5-¹²C; d₄] α-ketoglutarate. FIG. 3C provides representative stacked variable-temperature ¹³C NMR spectra of 5 mM [1-¹³C, 5-¹²C; d₄] α-ketoglutarate. FIG. 3D provides the corresponding ¹³C NMR spectrum of thermally polarized neat [1-¹³C]acetic acid. FIG. 3E provides a graph showing the buildup and decay of total ¹³C polarization of ¹³C-1 (i.e., integrating over all bound and free resonances) in [1-¹³C, 5-¹²C; d₄] α-ketoglutarate at B_{T}=0.42 μT. FIG. 3F provides a graph showing the corresponding ¹³C-1 T₁ relaxation curves at the Earth's field and clinically relevant 1.4 T field of the benchtop spectrometer. FIG. 3G provides a graph showing the total ¹³C polarization of ¹³C-1 [1-¹³C, 5-¹²C; d₄] α-ketoglutarate as a function of temperature. FIG. 3H provides a graph showing the total ¹³C polarization of ¹³C-1 [1-¹³C, 5-¹²C; d₄] α-ketoglutarate as a function of magnetic transfer field.

FIGS. 4A-4G provide the results of the SABRE-SHEATH hyperpolarization process for natural abundance disodium α-ketoglutarate. FIG. 4A is a schematic showing the SABRE-SHEATH hyperpolarization process and relevant polarization transfer path of disodium α-ketoglutarate. FIG. 4B provides a representative ¹³C NMR spectrum of a 5.6 mM solution of natural-bundance disodium α-ketoglutarate obtained by performing SABRE-SHEATH at +10° C. in CD₃OD. FIG. 4C provides the corresponding ¹³C NMR spectrum of thermally polarized neat [1-¹³C]acetic acid. FIG. 4D provides a graph showing the buildup and decay of total ¹³C polarization (i.e., integrating over all bound and free resonances) in disodium α-ketoglutarate at B_{T}=0.42 μT and T_{T}=+10° C. FIG. 4E provides a graph showing the total (bound+free)¹³C polarization decay of disodium α-ketoglutarate at the Earth's field and clinically relevant 1.4 T field of the benchtop spectrometer. FIG. 4F provides a graph showing the total ¹³C polarization of ¹³C-1 in natural abundance disodium α-ketoglutarate as a function of temperature. FIG. 4G provides a graph showing the total ¹³C polarization of ¹³C-1 in natural abundance disodium α-ketoglutarate as a function of magnetic transfer field.

### DETAILED DESCRIPTION OF THE INVENTION

In accordance with an aspect, the present invention provides a compound of Formula (I):

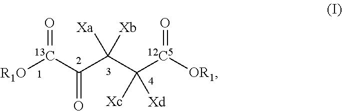

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

For example, the compound of Formula (I) may be one of the following:
(i) wherein one of Xa and Xb is deuterium and the other is hydrogen and both Xc and Xd are hydrogen as shown in Formula (Ia):

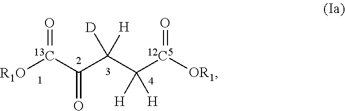

or a pharmaceutically acceptable salt thereof,
(ii) wherein one of Xc and Xd is deuterium and the other is hydrogen and both Xa and Xb are hydrogen as shown in Formula (Ib):

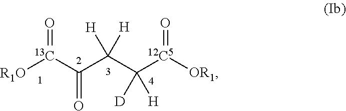

or a pharmaceutically acceptable salt thereof,
(iii) wherein one of Xa and Xb is deuterium and the other is hydrogen and one of Xc and Xd is deuterium and the other is hydrogen as shown in Formula (Ic):

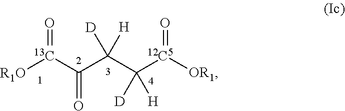

or a pharmaceutically acceptable salt thereof,
(iv) wherein both Xa and Xb are deuterium and both Xc and Xd are hydrogen as shown in Formula (Id):

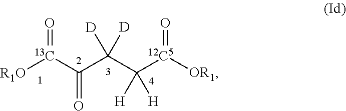

or a pharmaceutically acceptable salt thereof,
(v) wherein both Xa and Xb are hydrogen and both Xc and Xd are deuterium as shown in Formula (Ie):

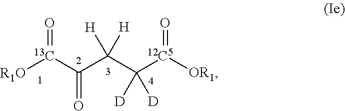

or a pharmaceutically acceptable salt thereof,
(vi) wherein both Xa and Xb are deuterium and one of Xc and Xd is deuterium and the other is hydrogen as shown in Formula (If):

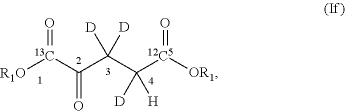

or a pharmaceutically acceptable salt thereof,
(vii) wherein both Xc and Xd are deuterium and one of Xa and Xb is deuterium and the other is hydrogen as shown in Formula (Ig):

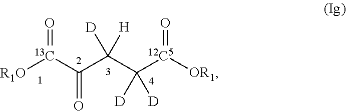

or a pharmaceutically acceptable salt thereof, or
(viii) wherein Xa, Xb, Xc, and Xd are deuterium as shown in Formula (Ih):

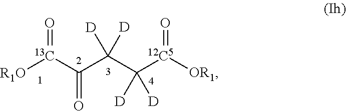

or a pharmaceutically acceptable salt thereof.

In any of the aspects of the compound of Formula (I) described herein (e.g., Formulae (Ia)-(Ih)), each R₁ may be independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl. In some embodiments, each R₁ is independently selected from a C₁-C₆ alkyl, for example, each R₁ can be methyl, ethyl, propyl (e.g., isopropyl or n-propyl), butyl (e.g., isobutyl, n-butyl, tert-butyl, or sec-butyl), pentyl, or hexyl. In some embodiments, each R₁ is independently selected from hydrogen, deuterium, and a cation. In embodiments where R₁ is a cation, it will be readily understood by a person of ordinary skill in the art that the compound of Formula (I) (e.g., Formulae (Ia)-(Ih)) is a salt (e.g., a pharmaceutically acceptable salt) where the negative charge on oxygen is balanced by the cation.

In another aspect, the present invention provides a composition comprising an isotopic mixture of a compound of Formula (I):

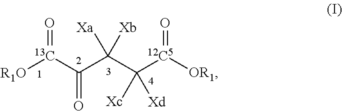

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, and wherein at least 25% of a sum total deuterium and hydrogen atoms defined by Xa+Xb+Xc+Xd are deuterium,
or a pharmaceutically acceptable salt thereof. In other words, the invention provides an isotopic mixture of a compound of Formula (I), wherein, on average, at least one of Xa+Xb+Xc+Xd is deuterium. When referring to a composition comprising an isotopic mixture, it will be understood that each compound in the composition comprising the isotopic mixture, aside from the isotopic labeling, is structurally identical, i.e., each R₁ is consistently defined. All other aspects of the compound of Formula (I) (e.g., Formulae (Ia)-(Ih)) in the composition comprising the isotopic mixture are as described herein.

In some embodiments of the isotopic mixture of the compound of Formula (I), at least 25% of a sum total deuterium and hydrogen atoms defined by Xa+Xb+Xc+Xd are deuterium. For example, in the compound of Formula (I), Xa can be hydrogen or deuterium, Xb can be hydrogen or deuterium, Xc can be hydrogen or deuterium, and Xd can be hydrogen or deuterium and the isotopic mixture of the compound of Formula (I) can have any combination of labeling such that on average, at least one of Xa, Xb, Xc, and Xd are deuterium. In some embodiments of the isotopic mixture of the compound of Formula (I), at least 50% of a sum total deuterium and hydrogen atoms defined by Xa+Xb+Xc+Xd are deuterium. In other words, the isotopic mixture of the compound of Formula (I) can have any combination of labeling such that on average, at least two of Xa, Xb, Xc, and Xd are deuterium. In other embodiments of the isotopic mixture of the compound of Formula (I), at least 75% of a sum total deuterium and hydrogen atoms defined by Xa+Xb+Xc+Xd are deuterium. In other words, the isotopic mixture of the compound of Formula (I) can have any combination of labeling such that on average, at least three of Xa, Xb, Xc, and Xd are deuterium. In certain embodiments of the isotopic mixture of the compound of Formula (I), at least 95% of a sum total deuterium and hydrogen atoms defined by Xa+Xb+Xc+Xd are deuterium. In other words, the isotopic mixture of the compound of Formula (I) can have any combination of labeling such that on average, all, or almost all of Xa, Xb, Xc, and Xd are deuterium. Thus, the composition comprising an isotopic mixture of a compound of Formula (I) can have from 25% to 100% (e.g., from 25% to 95%, from 25% to 75%, from 25% to 50%, from 50% to 100%, from 50% to 95%, from 50% to 75%, from 75% to 100%, or from 75% to 95%) of a sum total deuterium and hydrogen atoms defined by Xa, Xb, Xc, and Xd being deuterium.

In some embodiments of the isotopic mixture of the compound of Formula (I), at least 50% of a sum total deuterium and hydrogen atoms defined by Xa+Xb are deuterium. For example, in the compound of Formula (I), Xa can be hydrogen or deuterium and Xb can be hydrogen or deuterium and the isotopic mixture of the compound of Formula (I) can have any combination of labeling such that on average, at least one of Xa and Xb is deuterium. In some embodiments of the isotopic mixture of the compound of Formula (I), at least 75% of a sum total deuterium and hydrogen atoms defined by Xa and Xb are deuterium. In other embodiments of the isotopic mixture of the compound of Formula (I), at least 95% of a sum total deuterium and hydrogen atoms defined by Xa+Xb are deuterium. In other words, the isotopic mixture of the compound of Formula (I) can have any combination of labeling such that on average, all, or almost all of Xa and Xb are deuterium. Thus, the composition comprising an isotopic mixture of a compound of Formula (I) can have from 50% to 100% (e.g., from 50% to 95%, from 50% to 75%, from 75% to 100%, or from 75% to 95%) of a sum total deuterium and hydrogen atoms defined by Xa and Xb being deuterium.

Alternatively, or additionally, in some embodiments of the isotopic mixture of the compound of Formula (I), at least 50% of a sum total deuterium and hydrogen atoms defined by Xc+Xd are deuterium. For example, in the compound of Formula (I), Xc can be hydrogen or deuterium and Xd can be hydrogen or deuterium and the isotopic mixture of the compound of Formula (I) can have any combination of labeling such that on average, at least one of Xc and Xd are deuterium. In some embodiments of the isotopic mixture of the compound of Formula (I), at least 75% of a sum total deuterium and hydrogen atoms defined by Xc and Xd are deuterium. In other embodiments of the isotopic mixture of the compound of Formula (I), at least 95% of a sum total deuterium and hydrogen atoms defined by Xc+Xd are deuterium. In other words, the isotopic mixture of the compound of Formula (I) can have any combination of labeling such that on average, all, or almost all of Xc and Xd are deuterium. Thus, the composition comprising an isotopic mixture of a compound of Formula (I) can have from 50% to 100% (e.g., from 50% to 95%, from 50% to 75%, from 75% to 100%, or from 75% to 95%) of a sum total deuterium and hydrogen atoms defined by Xc and Xd being deuterium.

The present invention further provides a pharmaceutical composition comprising a hyperpolarized compound of Formula (I) (e.g., an effective amount of a hyperpolarized compound of Formula (I)), or a pharmaceutically acceptable salt thereof, and a pharmaceutically acceptable carrier. All aspects with respect to the compound of Formula (I), and the hyperpolarization thereof, are as described herein.

The present invention further provides a pharmaceutical composition comprising a hyperpolarized composition comprising an isotopic mixture of a compound of Formula (I) (e.g., an effective amount of an isotopic mixture of a compound of Formula (I)), or a pharmaceutically acceptable salt thereof, and a pharmaceutically acceptable carrier. As used herein, the phrase “hyperpolarized composition comprising an isotopic mixture of a compound of Formula (I)” refers to a composition comprising an isotopic mixture of a compound of Formula (I), wherein one or more of the isotopes are hyperpolarized. In other words, the hyperpolarized composition comprising an isotopic mixture of a compound of Formula (I) comprises a hyperpolarized isotopic mixture of a compound of Formula (I). All aspects with respect to the compound of Formula (I), the isotopic mixture, and the hyperpolarization thereof, are as described herein.

The present invention further provides a method of diagnosing or monitoring a patient having or suspected to have a cancer, the method comprising administering a hyperpolarized ketoglutarate compound or a pharmaceutical composition as described above and diagnosing or monitoring the patient by hyperpolarized ¹³C-MRI. For example, a hyperpolarized compound of Formula (I) (e.g., an effective amount of a hyperpolarized compound of Formula (I)) or a hyperpolarized composition comprising an isotopic mixture of a compound of Formula (I) (e.g., an effective amount of an isotopic mixture of a compound of Formula (I)), or a pharmaceutical composition comprising the same, can be used in the method of diagnosing or monitoring a patient having or suspected to have a cancer. For example, in some embodiments, the invention provides use of a hyperpolarized compound of Formula (I) (e.g., an effective amount of a hyperpolarized compound of Formula (I)) or a hyperpolarized composition comprising an isotopic mixture of a compound of Formula (I) (e.g., an effective amount of an isotopic mixture of a compound of Formula (I)), or a pharmaceutical composition comprising the same, in a method of diagnosing or monitoring a patient having or suspected to have a cancer, the method comprising diagnosing or monitoring the patient by hyperpolarized ¹³C-MRI. In some embodiments, the method or use comprises identifying a mutation or mutations responsible for the cancer. In certain embodiments, the method or use identifies an IDH1 mutation as being responsible for the cancer. In other words, the method or use can be used to identify whether the patient has a tumor, for example, an IDH1 mutation.

The present invention further provides a method of preparing a compound of Formula (I) (e.g., Formulae (Ia)-(Ih)) or a pharmaceutically acceptable salt thereof, as described herein, wherein each R₁ is independently selected from hydrogen, deuterium, and a cation, the method comprising:
(i) reacting a compound of Formula (II) with deuterium oxide under basic conditions in an organic solvent and obtaining a compound of Formula (III):

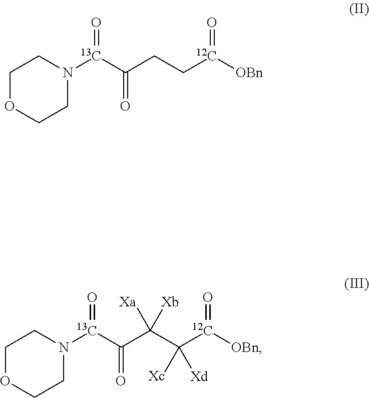

wherein Xa, Xb, Xc, and Xd are each independently hydrogen or deuterium, provided that at least one of Xa, Xb, Xc, and Xd is deuterium, and wherein Bn is benzyl; and
(ii) reacting the compound of Formula (III) with DCl and D₂O or HCl and H₂O to obtain the compound of Formula (I) (e.g., Formulae (Ia)-(Ih)) or a pharmaceutically acceptable salt thereof. All other aspects of the compound of Formula (I) are as described herein. In some embodiments, each R₁ is hydrogen and the method comprises reacting the compound of Formula (III) with HCl and H₂O. In some embodiments, each R₁ is deuterium and the method comprises reacting the compound of Formula (III) with DCl and D₂O. In some embodiments, each R₁ is a cation and the method further comprises (iii) reacting the compound of Formula (I) with a base.

The present invention further provides a method of preparing an isotopic mixture of a compound of Formula (I), as described herein, wherein each R₁ is independently selected from hydrogen, deuterium, and a cation, the method comprising:
(i) reacting a compound of Formula (II) with deuterium oxide under basic conditions in an organic solvent and obtaining an isotopic mixture of a compound of Formula (III):

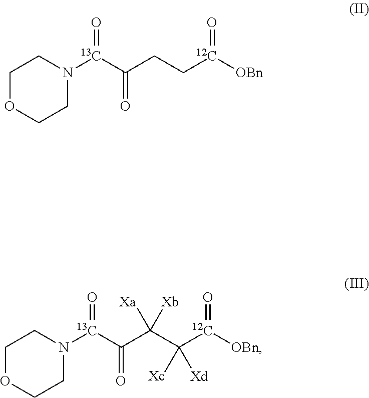

wherein Xa, Xb, Xc, and Xd are each independently hydrogen or deuterium, and wherein at least 25% of a sum total deuterium and hydrogen atoms defined by Xa+Xb+Xc+Xd are deuterium; and
(ii) reacting the isotopic mixture of the compound of Formula (III) with DCl and D₂O or HCl and H₂O to obtain the isotopic mixture of the compound of Formula (I) or a pharmaceutically acceptable salt thereof. All other aspects of the isotopic mixture of the compound of Formula (I) are as described herein. In some embodiments, each R₁ is hydrogen and the method comprises reacting the compound of Formula (III) with HCl and H₂O. In some embodiments, each R₁ is deuterium and the method comprises reacting the compound of Formula (III) with DCl and D₂O. In some embodiments, each R₁ is a cation and the method further comprises (iii) reacting the compound of Formula (I) with a base.

The present invention further provides a method of preparing a compound of Formula (I) (e.g., Formulae (Ia)-(Ih)) or a pharmaceutically acceptable salt thereof, as described herein, wherein each R₁ is independently selected from hydrogen and a cation, the method comprising:
(i) reacting a compound of Formula (II) with deuterium oxide under basic conditions in an organic solvent and obtaining a compound of Formula (III):

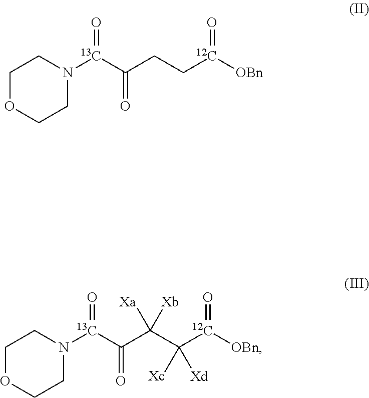

wherein Xa, Xb, Xc, and Xd are each independently hydrogen or deuterium, provided that at least one of Xa, Xb, Xc, and Xd is deuterium, and wherein Bn is benzyl; and
(ii) reacting the compound of Formula (III) with HCl and H₂O to obtain the compound of Formula (I) (e.g., Formulae (Ia)-(Ih)) or a pharmaceutically acceptable salt thereof. All other aspects of the compound of Formula (I) are as described herein. In some embodiments, each R₁ is a cation and the method further comprises (iii) reacting the compound of Formula (I) with a base.

The present invention further provides a method of preparing an isotopic mixture of a compound of Formula (I), as described herein, wherein each R₁ is independently selected from hydrogen and a cation, the method comprising:
(i) reacting a compound of Formula (II) with deuterium oxide under basic conditions in an organic solvent and obtaining an isotopic mixture of a compound of Formula (III):

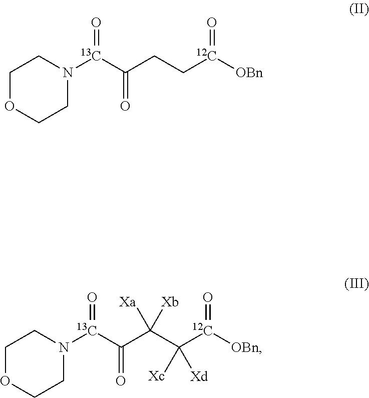

wherein Xa, Xb, Xc, and Xd are each independently hydrogen or deuterium, and wherein at least 25% of a sum total deuterium and hydrogen atoms defined by Xa+Xb+Xc+Xd are deuterium; and
(ii) reacting the isotopic mixture of the compound of Formula (III) with HCl and H₂O to obtain the isotopic mixture of the compound of Formula (I) or a pharmaceutically acceptable salt thereof. All other aspects of the isotopic mixture of the compound of Formula (I) are as described herein. In some embodiments, each R₁ is a cation and the method further comprises (iii) reacting the compound of Formula (I) with a base.

The present invention further provides a method of preparing a compound of Formula (D):

comprising:

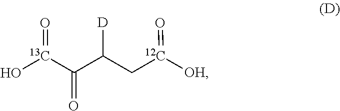 [SMILES: [2H]C(C[12C](=O)O)C(=O)[13C](=O)O]

(i) epoxidizing a compound of Formula (A) to obtain an epoxy compound of Formula (B):

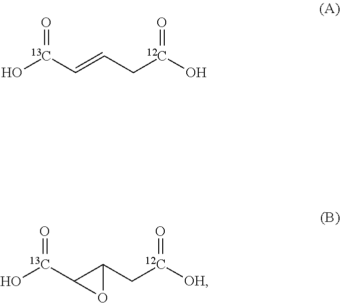 [SMILES: O=[12C](O)C/C=C/[13C](=O)O ; O=[12C](O)CC1OC1[13C](=O)O]

(ii) deuterating the compound of Formula (B) to obtain a compound of Formula (C):

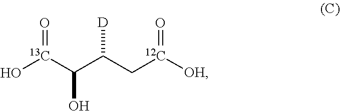 [SMILES: [2H][C@H](C[12C](=O)O)[C@@H](O)[13C](=O)O]

(iii) oxidizing the compound of Formula (C) in an alkaline solution to obtain the compound of Formula (D). In some embodiments, the compound of Formula (B) is deuterated by reacting with NaBD₄ in a solution of D₂O and lithium bromide. Alternatively,
or additionally, in some embodiments, the alkaline solution in (iii) comprises 4-(dimethylamino)pyridine and sodium bicarbonate.

The present invention further provides a method of preparing a compound of Formula (L):

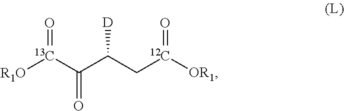

wherein each R₁ is independently H or alkyl, the method comprising:
(i) reacting a compound of Formula (E) with tert-butyldimethylsilyl chloride (TBSCl) bonded to imidazole, followed by reaction with catecholborane and with water to obtain a compound of Formula (F):

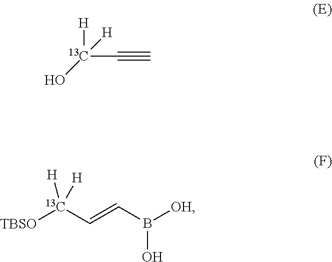

(ii) esterifying the compound of Formula (F) to obtain a compound of Formula (G) by reacting the compound of Formula (F) with a bromomethyl ester having ¹²C ester carbonyl group in the presence of tris(dibenzylideneacetone) dipalladium ligand in a basic medium to obtain a compound of Formula (G), wherein R₁ is H or alkyl:

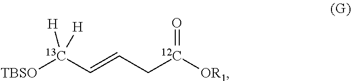

(iii) reacting the compound of Formula (G) with tetrabutylammonium fluoride to obtain a compound of Formula (H):

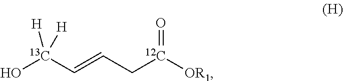

(iv) epoxidizing the compound of Formula (H) to obtain a compound of Formula (J):

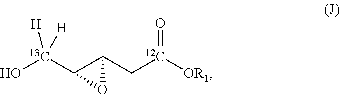

(v) deuterating the compound of Formula (J) by reacting the compound of Formula (J) with lithium/copper deuteride to obtain a compound of Formula (K):

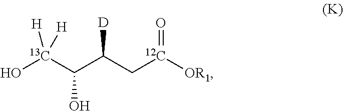

(vi) oxidizing the compound of Formula (K) to obtain the compound of Formula (L). In some embodiments, the epoxidation is carried out by reaction with an alkali metal tungstate and hydrogen peroxide. Alternatively, or additionally, in some embodiments, the compound of Formula (B) is deuterated by reaction with NaBD₄ in D₂O containing LiBr.

The present invention further provides a method of preparing a compound of Formula (L):

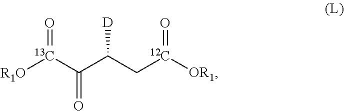

wherein R₁ is H or alkyl, the method comprising:
(i) reacting a compound of Formula (M) with trimethylsilyl trifluoromethanesulfonate and palladium (0) to obtain a compound of Formula (N):

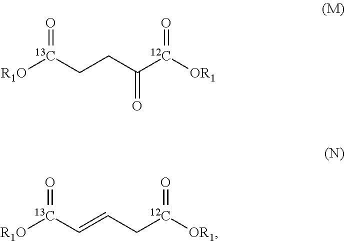

(ii) epoxidizing the compound of Formula (N) to obtain a compound of Formula (P):

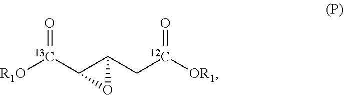

(iii) reacting the compound of Formula (P) with deuterated sodium borohydride in D₂O with lithium bromide to obtain a compound of Formula (Q):

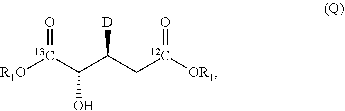

and
(iv) oxidizing the compound of Formula (Q) in an alkaline solution to obtain the compound of Formula (L). The compound of Formula (Q) can be oxidized by any suitable means to provide ketoglutarate. In some embodiments, the compound of Formula (Q) is oxidized through Dess-Martin oxidation to form ketoglutarate. Alternatively, or additionally, in some embodiments, the epoxidation of the compound of Formula (N) is carried out by reaction with a rare earth metal-biphenyldiol-Ph₃—As═O complex. Alternatively, or additionally, in some embodiments, the compound of Formula (B) is deuterated by reaction with NaBD₄ in D₂O containing LiBr. Alternatively, or additionally, in some embodiments, the alkaline solution in (iv) comprises 4-(dimethylamino)pyridine and sodium bicarbonate.

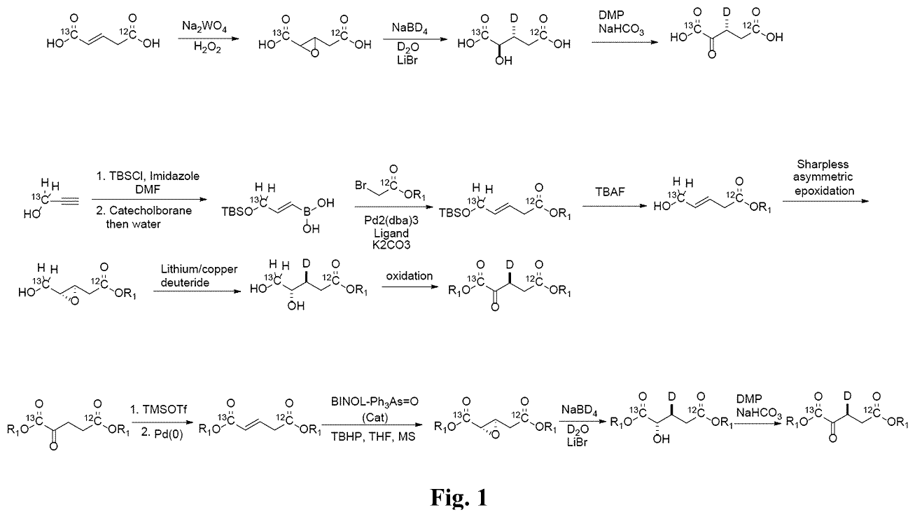

Exemplary epoxidation methods for the synthesis of mono-3-deuterated ketoglutarates in accordance with aspects of the invention are provided in FIG. 1.

The present invention further provides a method of preparing a hyperpolarized ketoglutarate compound comprising:
(i) providing a SABRE polarization transfer precatalyst;
(ii) combining the SABRE polarization transfer precatalyst with a ketoglutarate compound, parahydrogen, and optionally a co-ligand in a solvent to form a mixture comprising an active SABRE catalyst; and
(iii) hyperpolarizing the mixture comprising the active SABRE catalyst by exposing the mixture to a magnetic field or radiofrequency excitation to transfer the polarization from parahydrogen to the ketoglutarate compound to form the hyperpolarized ketoglutarate compound.

The ketoglutarate compound can be any suitable ketoglutarate compound regardless of the isotopic content. For example, the ketoglutarate compound can be deuterated, non-deuterated, or an isotopic mixture thereof. Generally, the ketoglutarate compound is ¹³C labeled, and, more particularly, ¹³C labeled at the C₁ position. In some embodiments, the ketoglutarate compound is a compound of Formula (I) or (IV):

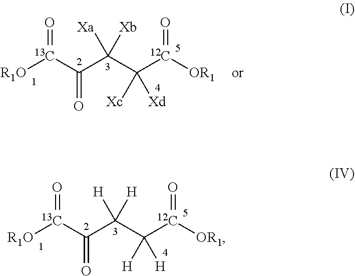

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

Each R₁ may be independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl. In some embodiments, each R₁ is independently selected from a C₁-C₆ alkyl, for example, each R₁ can be methyl, ethyl, propyl (e.g., isopropyl or n-propyl), butyl (e.g., isobutyl, n-butyl, tert-butyl, or sec-butyl), pentyl, or hexyl. In some embodiments, each R₁ is independently selected from hydrogen, deuterium, and a cation. In embodiments where R₁ is a cation, it will be readily understood by a person of ordinary skill in the art that the compound of Formula (I) (e.g., Formulae (Ia)-(Ih)) is a salt (e.g., a pharmaceutically acceptable salt) where the negative charge on oxygen is balanced by the cation. In certain embodiments, each R₁ independently is a cation or C₁-C₆ alkyl.

In some embodiments, the ketoglutarate compound is an isotopic mixture of a compound of Formula (I) described herein.

The method of preparing a hyperpolarized ketoglutarate compound comprises providing a SABRE polarization transfer precatalyst. The SABRE (signal amplification by reversible exchange) polarization transfer precatalyst can be any suitable SABRE polarization transfer precatalyst, many of which are known in the art. In some embodiments, the SABRE polarization transfer precatalyst comprises a d-block element such as, for example, Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, and/or Hg. In certain embodiments, the SABRE polarization transfer precatalyst comprises an element of group 9 of the periodic table, i.e., Co, Rh, Ir, or Mt. In preferred embodiments, the SABRE polarization transfer precatalyst comprises Ir or Co. For example, the SABRE polarization transfer precatalyst can be [Ir(COD)(IMes)(Cl)].

In some embodiments, the SABRE polarization transfer precatalyst may comprise one or more ligands such as, for example, a phosphine ligand, a carbene ligand, an imidazole ligand, and/or a pincer ligand. Alternatively, the SABRE polarization transfer precatalyst may comprise a tridentate chelating ligand. In some embodiments, the SABRE polarization transfer precatalyst comprises a phosphine ligand. Generally, phosphine ligands of the precatalysts are electron-rich and sterically demanding and give very high polarization transfer to pyridine protons. Examples of phosphine ligands include, but are not limited to the following.

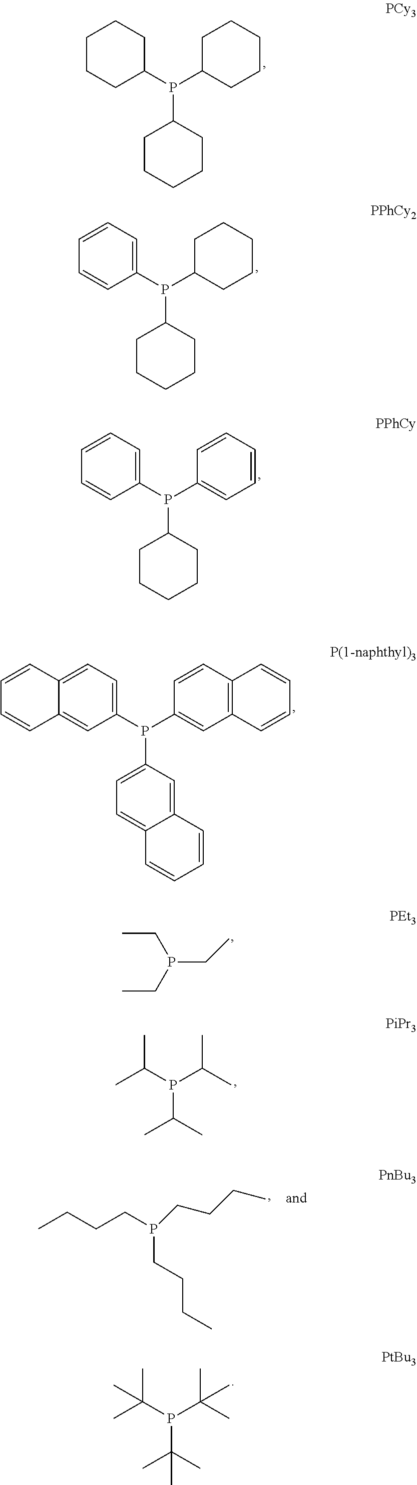

In some embodiments, the SABRE polarization transfer precatalyst comprises a carbene ligand. Generally, carbene ligands such as N-heterocyclic carbene (NHC) ligands can also give very high polarization levels and may even provide higher polarization than phosphine ligands due to the increased steric bulk as well as electron donating capacity of the NHCs. Another advantage is that they provide faster substrate dissociation. Examples of NHCs include, but are not limited to the following:

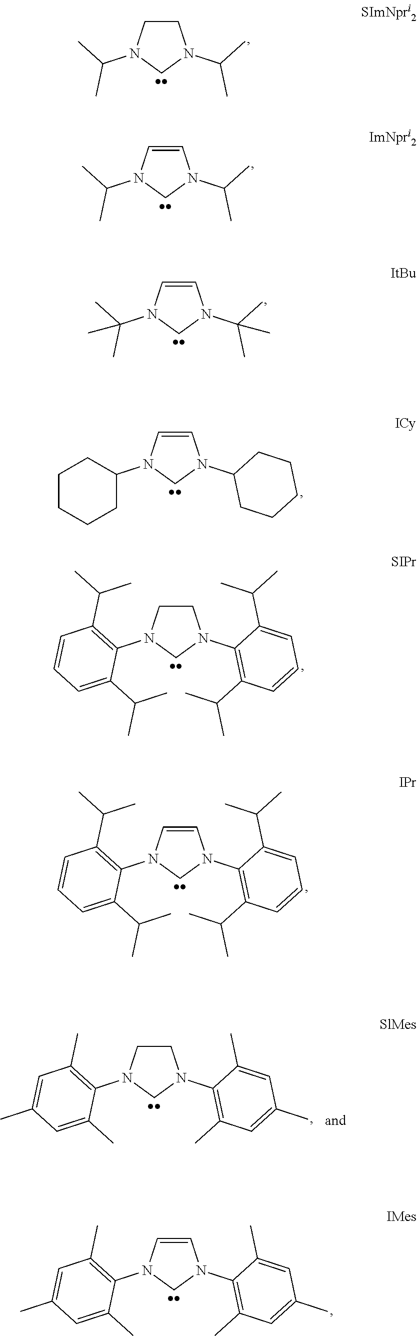

wherein IMes is 1,3-bis(2,4,6-trimethylphenyl)imidazole-2-ylidene.

Alternatives to IMes include substituted IMes variants with para, meta, and/or ortho substitution. Exemplary alternatives include, but are not limited to the following para-substituted IMes's:

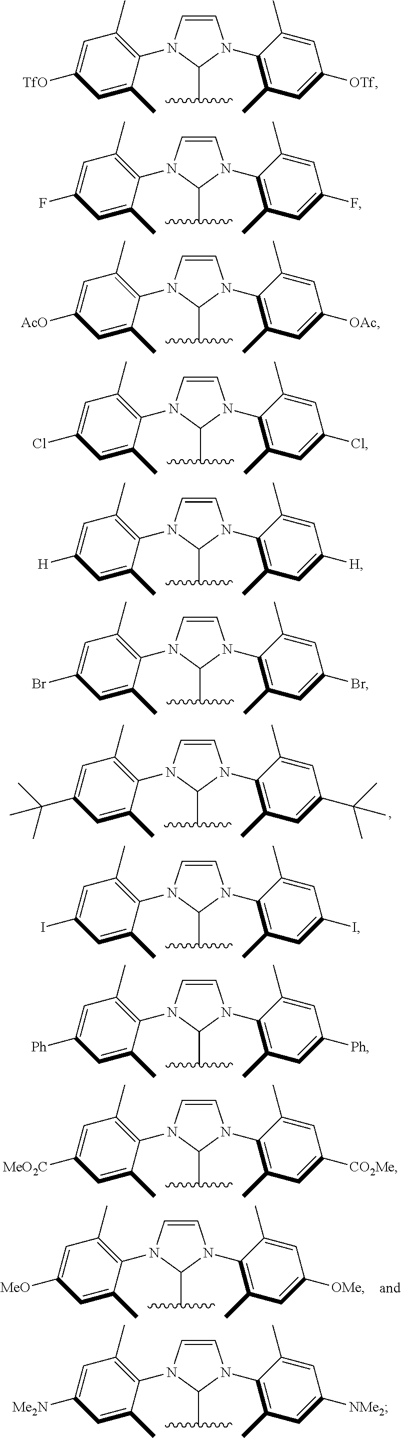

ortho-substituted IMes's:

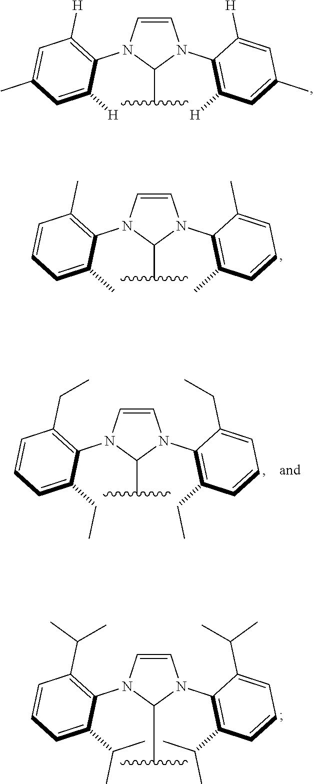

meta-substituted IMes:

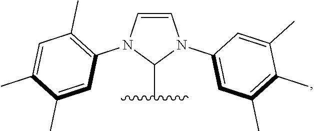

and other IMes's:

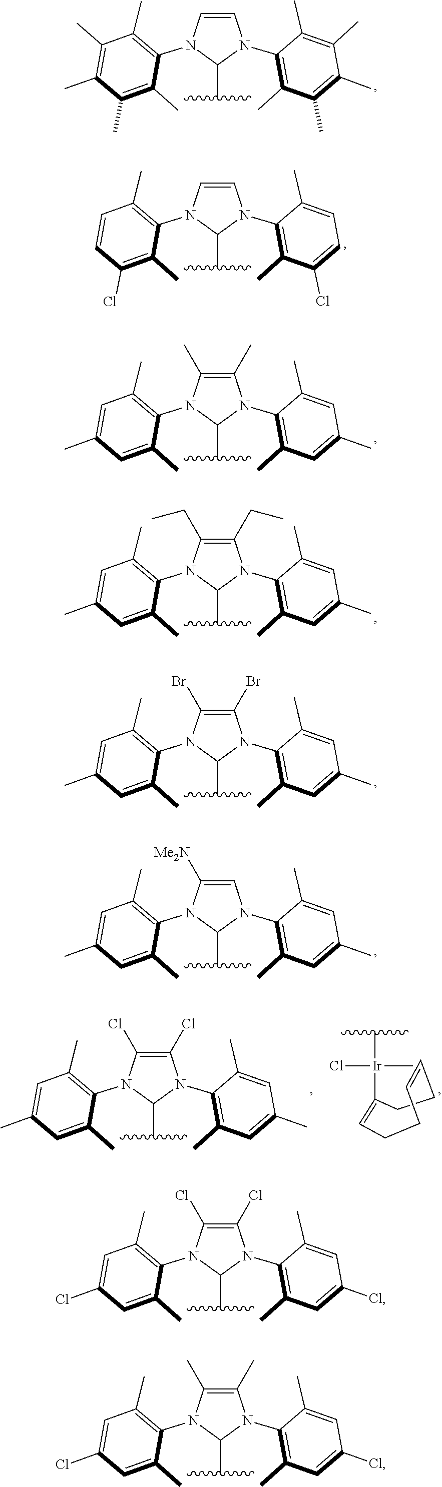

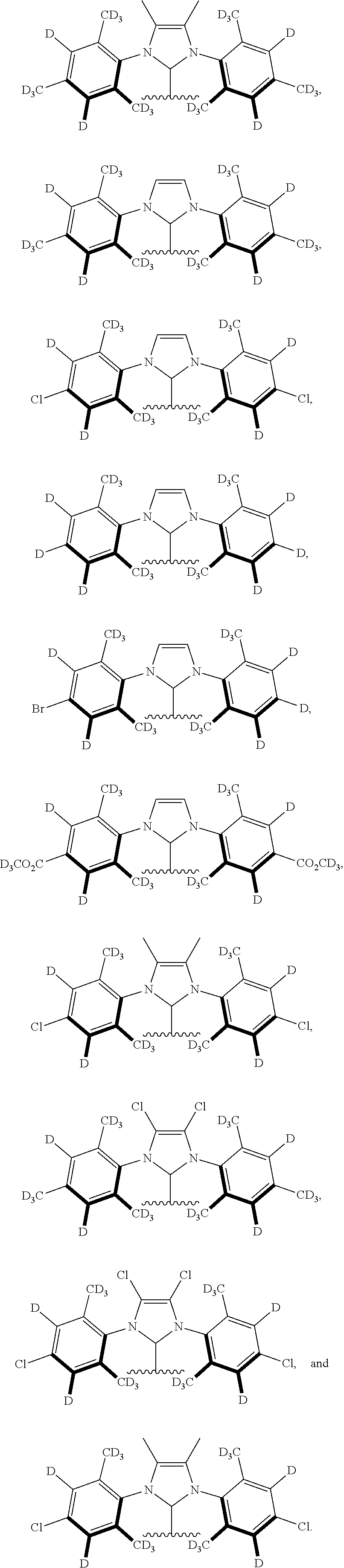

In some embodiments, the SABRE polarization transfer precatalyst comprises a pincer chelating ligand. Exemplary pincer chelating ligands include, but are not limited to the pincer chelating ligands shown in the following SABRE polarization transfer precatalysts:

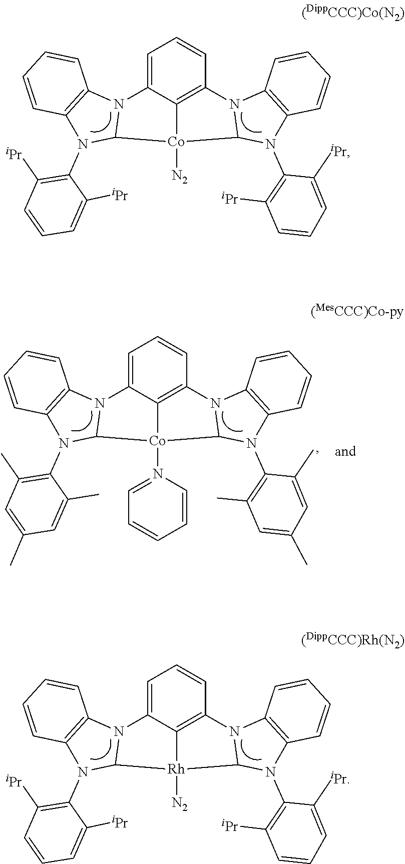

These SABRE polarization transfer precatalysts may also be suitable for the methods described herein.

In some embodiments, the SABRE catalyst can be prepared using a water soluble SABRE polarization transfer precatalyst. Water soluble SABRE polarization transfer precatalysts can be prepared by any suitable method. For example, water-soluble SABRE polarization transfer precatalysts may be prepared by adding polar groups to NHC ligands, including choline moieties, or including alkoxy (e.g., pegylated) groups. Exemplary water soluble SABRE polarization transfer precatalysts include, but are not limited to, the

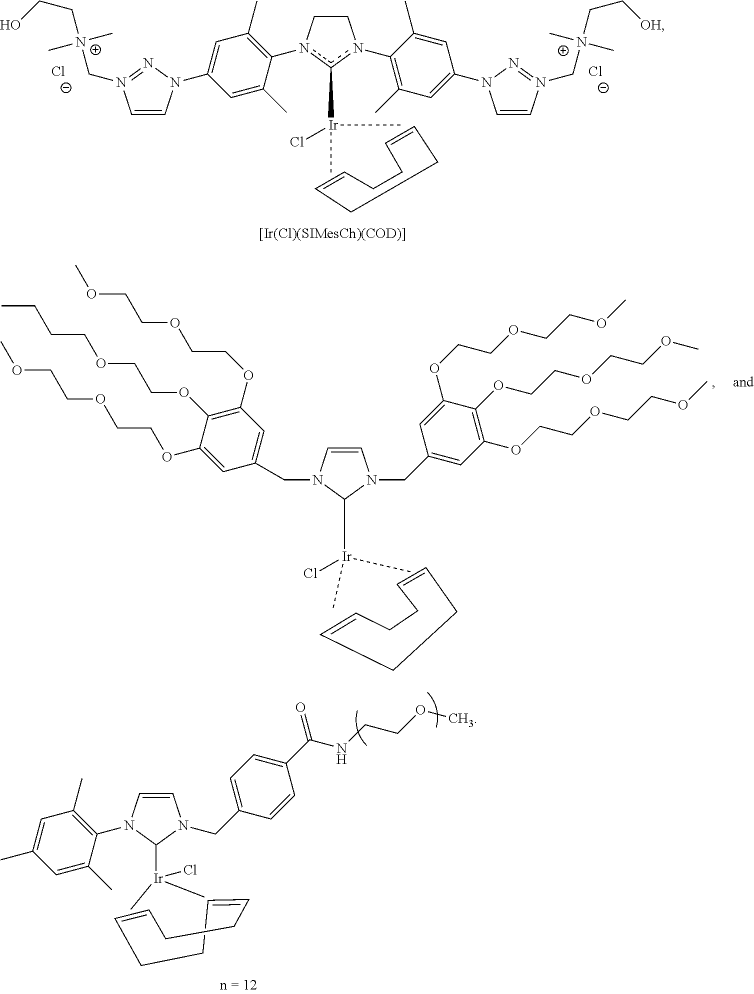

The SABRE polarization transfer precatalyst can also be a heterogeneous catalyst, which is suitable for in vivo applications. For example, the SABRE polarization transfer precatalyst can be an iridium catalyst, which is immobilized on a solid support such as, for example,

a polymer microbead:

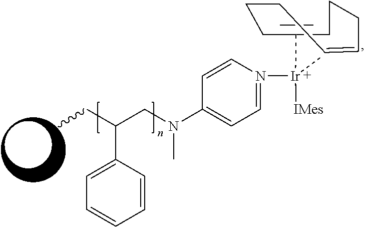

a core-shell nanoparticle (or nano-SABRE catalysts):

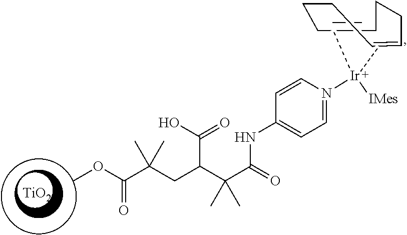

polymer combs:

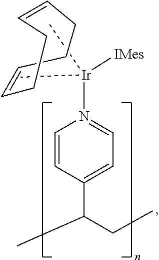

which are schematically shown below:

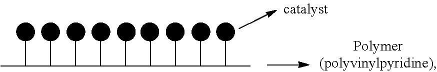 [SMILES: CC(C)C(C)C(C)C(C)C(C)C(C)C(C)C(C)C(C)C]

and silica gel bonded catalysts:

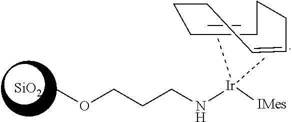

The active SABRE catalyst can be prepared by any suitable method. Generally, the active SABRE catalyst is prepared by combining the SABRE polarization transfer precatalyst with a ketoglutarate compound, parahydrogen, and optionally a co-ligand in a solvent to form a mixture comprising an active SABRE catalyst. In some embodiments, the active SABRE catalyst is prepared by combining the SABRE polarization transfer precatalyst with a ketoglutarate compound, parahydrogen, and a co-ligand in a solvent to form a mixture comprising an active SABRE catalyst. The co-ligand, when included in the preparation of the active SABRE catalyst, can be combined with the SABRE polarization transfer precatalyst in any order and by any suitable means. For example, the co-ligand, when included in the preparation of the active SABRE catalyst, can be provided first to interact with the transfer precatalyst to facilitate formation of the active SABRE catalyst. Alternatively, the co-ligand, when included in the preparation of the active SABRE catalyst, can be added together with the ketoglutarate compound to facilitate formation of the active SABRE catalyst. In some embodiments, the co-ligand, ketoglutarate compound, and parahydrogen are essentially combined with the SABRE polarization transfer precatalyst in the solvent at the same time to facilitate formation of the active SABRE catalyst. In other embodiments, the ketoglutarate compound is provided first to interact with the transfer precatalyst to facilitate formation of the active SABRE catalyst. In some embodiments, the co-ligand and the ketoglutarate compound are combined with the SABRE polarization transfer precatalyst in the solvent, and the parahydrogen is added to (e.g., bubbled through) the resulting mixture. In other embodiments, the ketoglutarate compound is combined with the SABRE polarization transfer precatalyst in the solvent, and the parahydrogen is added to (e.g., bubbled through) the resulting mixture.

In some embodiments, the active SABRE catalyst is prepared by combining the SABRE polarization transfer precatalyst with a co-ligand in addition to the ketoglutarate compound and parahydrogen. Any suitable co-ligand can be used. For example, the co-ligand can be a compound comprising a sulfoxide group. Examples of compounds comprising a sulfoxide group can be selected from the group consisting of DMSO, phenyl methyl sulfoxide, phenyl chloromethyl sulfoxide, diphenyl sulfoxide, dibenzoyl sulfoxide, and dibutyl sulfoxide.

The method can be performed in any suitable solvent. In some embodiments, the solvent comprises water (e.g., deionized water) and optionally one or more water-miscible organic solvents. In other embodiments, the solvent comprises one or more organic solvents. Examples of organic solvents that can be used in the method include alcohols such as propenyl alcohol, isopropyl alcohol, ethanol, 1-propanol, methanol, 1-hexanol, and the like; ketones such as acetone, diacetone alcohol, methyl ethyl ketone, and the like; esters such as ethyl formate, propyl formate, ethyl acetate, methyl acetate, methyl lactate, butyl lactate, ethyl lactate, and the like; ethers including sulfoxides such as dimethyl sulfoxide (DMSO), tetrahydrofuran, dioxane, diglyme, and the like; amides such as N,N-dimethylformamide, dimethylimidazolidinone, N-methylpyrrolidone, and the like; polyhydric alcohols and derivatives of the same such as ethylene glycol, glycerol, diethylene glycol, diethylene glycol monomethyl ether, and the like; and nitrogen-containing organic compounds such as acetonitrile, amylamine, isopropylamine, imidazole, dimethylamine, and the like. In some embodiments, the solvent is water, i.e., without the presence of an organic solvent. The solvent can be a deuterated solvent or a non-deuterated solvent. In some embodiments, the solvent is a deuterated solvent. In other embodiments, the solvent is a non-deuterated solvent.

The parahydrogen can be combined with the SABRE polarization transfer precatalyst by any suitable means. For example, the parahydrogen can be bubbled through the mixture comprising the SABRE polarization transfer precatalyst, the parahydrogen can be added as a gas blanket over the mixture comprising the SABRE polarization transfer precatalyst, or the mixture comprising the SABRE polarization transfer precatalyst can be placed under increased pressure of parahydrogen. In some embodiments, the parahydrogen is bubbled through the mixture comprising the SABRE polarization transfer precatalyst. For example, the active SABRE catalyst can be prepared by the addition of parahydrogen to the pre-catalyst in the presence of excess substrate. In some embodiments, the SABRE catalyst is prepared as follows:

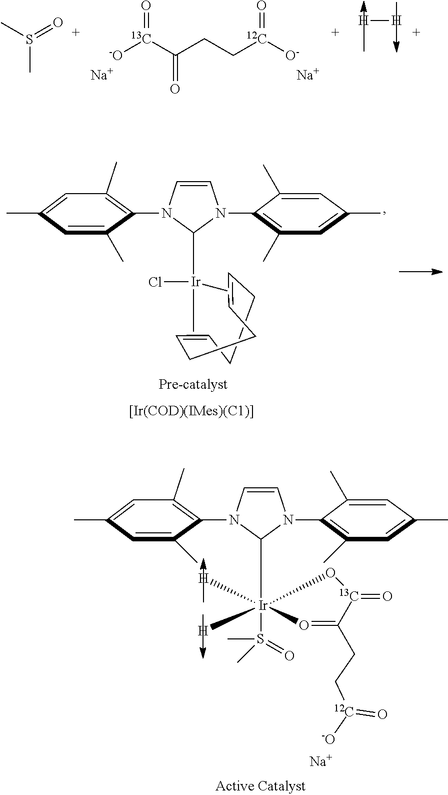

wherein IMes is 1,3-bis(2,4,6-trimethylphenyl)imidazole-2-ylidene.

As a result, the active SABRE (signal amplification by reversible exchange) catalyst comprises at least a d-block element such as, for example, Sc, Ti, V, Cr, Mn, Fe, Co, Ni, Cu, Zn, Y, Zr, Nb, Mo, Tc, Ru, Rh, Pd, Ag, Cd, L a, Hf, Ta, W, Re, Os, Ir, Pt, Au, and/or Hg, parahydrogen, and the ketoglutarate compound. In certain embodiments, the active SABRE catalyst comprises an element of group 9 of the periodic table, i.e., Co, Rh, Ir, or Mt. In preferred embodiments, the active SABRE catalyst comprises Ir or Co. In some embodiments, the active SABRE catalyst includes the ligand, which was present on the SABRE polarization transfer precatalyst. For example, the active SABRE catalyst may include a phosphine ligand, a carbene ligand, an imidazole ligand, and/or a pincer ligand.

In some embodiments, the active SABRE catalyst includes a co-ligand. Any suitable co-ligand can be used. For example, the co-ligand can be a compound comprising a sulfoxide group. Examples of compounds comprising a sulfoxide group can be selected from the group consisting of DMSO, phenyl methyl sulfoxide, phenyl chloromethyl sulfoxide, diphenyl sulfoxide, dibenzoyl sulfoxide, and dibutyl sulfoxide.

Thus, in some embodiments, the active SABRE catalyst comprises at least a d-block element (e.g., Ir or Co), parahydrogen, the ketoglutarate compound, and the co-ligand. In other embodiments, the active SABRE catalyst comprises at least a d-block element (e.g., Ir or Co), parahydrogen, the ketoglutarate compound, the co-ligand, and one or more ligands.

In some embodiments, the active SABRE catalyst comprises the salt of the compound of Formula (I), which is subjected to a polarization transfer to obtain a hyperpolarized compound. Examples of the active SABRE catalyst are:

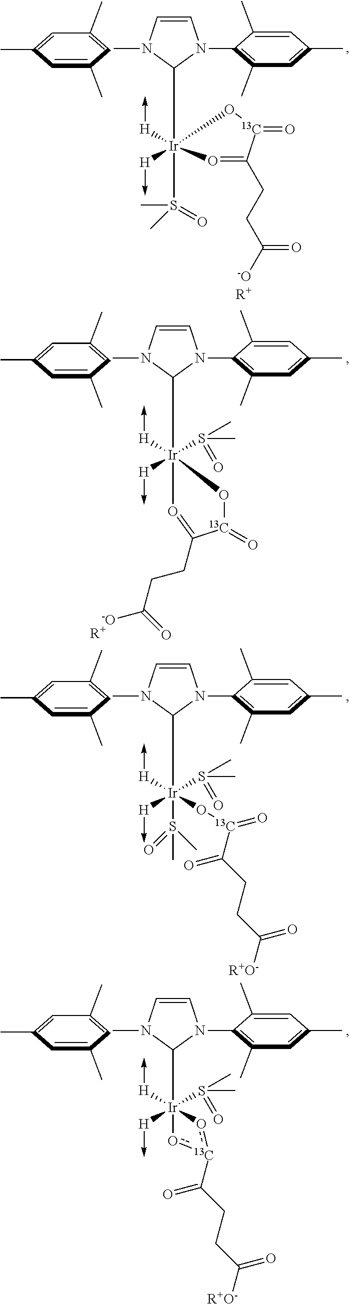

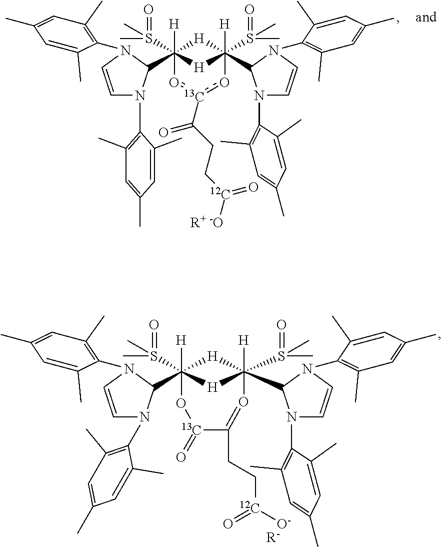

wherein R is a cation (e.g., Na⁺) described herein.

The methods of preparing a hyperpolarized ketoglutarate, described herein, comprise hyperpolarizing the mixture comprising the active SABRE catalyst by exposing the mixture to a magnetic field or radiofrequency excitation to transfer the polarization from parahydrogen to the ketoglutarate compound to form the hyperpolarized ketoglutarate compound. Initially, the hyperpolarized ketoglutarate compound is complexed with the hyperpolarized SABRE catalyst; however, it will be understood by a person of ordinary skill in the art that the hyperpolarized ketoglutarate compound can be replaced by another ketoglutarate compound molecule such that the process can be repeated and the free hyperpolarized ketoglutarate compound is produced.

The transfer of polarization from parahydrogen to the ketoglutarate compound to form the hyperpolarized ketoglutarate compound can occur under any suitable magnetic field or radiofrequency excitation. For example, the transfer of polarization from parahydrogen to a carbon atom of the ketoglutarate compound can occur at a magnetic field below the magnetic field of earth. The suitable level of magnetic field or radiofrequency excitation necessary to transfer the polarization from parahydrogen to the ketoglutarate compound to form the hyperpolarized ketoglutarate compound will be readily apparent to a person of ordinary skill in the art.

In some embodiments, the method comprises replenishing the parahydrogen in the mixture during the step of hyperpolarizing the mixture comprising the active SABRE catalyst by exposing the mixture to a magnetic field or radiofrequency excitation to transfer the polarization from parahydrogen to the ketoglutarate compound to form the hyperpolarized ketoglutarate compound. In other words, in some embodiments, the method comprises bubbling parahydrogen through the mixture comprising the active SABRE catalyst during the step of hyperpolarizing the mixture comprising the active SABRE catalyst by exposing the mixture to a magnetic field or radiofrequency excitation to transfer the polarization from parahydrogen to the ketoglutarate compound to form the hyperpolarized ketoglutarate compound.

In some embodiments, the hyperpolarized ketoglutarate compound is of Formula (HP-I) or (HP-IV)

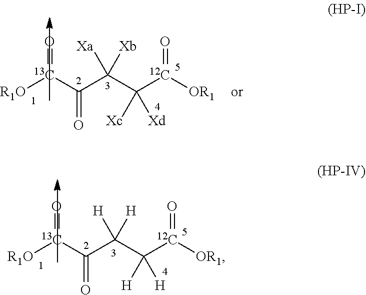

wherein the arrow in Formula (HP-I) and Formula (IP-IV) designates the hyperpolarization,
wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

In the present application, the term “alkyl” refers to a saturated hydrocarbon group, having the specified number of carbon atoms, usually from 1 to about 6 carbon atoms. Examples of alkyl groups include methyl, ethyl, propyl (e.g., isopropyl or n-propyl), butyl (e.g., isobutyl, n-butyl, tert-butyl, or sec-butyl), pentyl, or hexyl.

In the present application, the term “cycloalkyl” refers to a saturated hydrocarbon ring group, having the specified number of carbon atoms, usually from 3 to about 7 carbon atoms. Examples of cycloalkyl groups include cyclopropyl, cyclobutyl, cyclopentyl, or cyclohexyl as well as bridged or caged saturated ring groups such as norbornane or adamantane.

In the present application, the term “cycloalkyl” refers to a saturated hydrocarbon ring group, having the specified number of carbon atoms, usually from 3 to about 7 carbon atoms. Examples of cycloalkyl groups include cyclopropyl, cyclobutyl, cyclopentyl, or cyclohexyl as well as bridged or caged saturated ring groups such as norbornane or adamantane.

The term “aryl” refers to a stable monocyclic or polycyclic, substituted or unsubstituted aromatic ring having 5 to 60 ring carbon atoms, e.g., phenyl, tolyl, xylyl, naphthyl, phenanthryl, and anthracenyl.

The term “heteroaryl” refers to a stable monocyclic aromatic ring having the indicated number of ring atoms which contains from 1 to 3, or in some aspects, from 1 to 2, heteroatoms chosen from N, O, and S, with remaining ring atoms being carbon, or a stable bicyclic or tricyclic system containing at least one 5- to 7-membered aromatic ring which contains from 1 to 3, or in some aspects, from 1 to 2, heteroatoms chosen from N, O, and S, with remaining ring atoms being carbon. Monocyclic heteroaryl groups typically have from 5 to 7 ring atoms, in some aspects, bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Heteroaryl groups include, but are not limited to, oxazolyl, piperazinyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, henzofrijoxazolyl, benzofuranyl, benzothiazolyl, benzolhiophenyl, benzoxadiazolyl, dihydrobenzodioxynyl, furanyl, imidazolyl, indolyl, isothiazolyl, and isoxazolyl.

The term “heterocyclyl” refers to a saturated or unsaturated cyclic group having the indicated number of ring atoms containing from 1 to about 3 heteroatoms chosen from N, O, and S, with remaining ring atoms being carbon. Examples of heterocyclyl groups include piperazine and thiazole groups.

The term “heterocycloalkyl” refers to a saturated cyclic group having the indicated number of ring atoms containing from 1 to about 3 heteroatoms chosen from N, O, and 8, with remaining ring atoms being carbon. Examples of heterocycloalkyl groups include tetrahydrofuranyl and pyrrolidinyl groups.

The term “pharmaceutical composition” refers to a composition comprising at least one active agent, such as a compound or salt of Formula (I), and at least one other substance, such as a carrier, i.e., a diluent, excipient, or vehicle with which an active compound is administered. A “pharmaceutically acceptable carrier” refers to a substance, e.g., excipient, diluent, or vehicle, that is useful in preparing a pharmaceutical composition that is generally safe, non-toxic and neither biologically nor otherwise undesirable, and includes a carrier that is acceptable tor veterinary use as well as human pharmaceutical use. A “pharmaceutically acceptable carrier” includes both one and more than one such carrier.

The term “patient” refers to a human or non-human animal in need of medical treatment. Medical treatment can include treatment of an existing condition, such as a disease or disorder or diagnostic treatment. In some embodiments the patient is a human patient.

The term “treatment” or “treating” refers to providing an active compound to a patient in an amount sufficient to measurably reduce any cancer symptom, slow cancer progression or cause cancer regression. In certain embodiments treatment of the cancer may be commenced before the patient presents symptoms of the disease.

Compounds of Formula (I) may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Pharmaceutically acceptable salts” include derivatives of the disclosed compounds in which the parent compound is modified by making inorganic and organic, nontoxic, acid or base addition salts thereof. The salts of the compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)n-COOH where n is 0-4, and the like. Lists of additional suitable salts may be found, e.g., in G, Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth Editors, Wiley-VCH, 2002. The composition may further include at least one pharmaceutically acceptable excipient. A pharmaceutically acceptable excipient, as used herein, refers to a non-active pharmaceutical ingredient (“API”) substance such as a disintegrator, a binder, a filler, and a lubricant used in formulating pharmaceutical products. Each of these substances is generally safe for administering to humans according to established governmental standards, including those promulgated by the United States Food and Drug Administration (“FDA”).

As used herein the term “cation” refers to any ion with a positive charge capable of balancing a negative charge on the compound of Formula (I). Generally, such cations are incorporated into the salts (e.g., the pharmaceutically acceptable salts) of the compound of Formula (I) by reacting free acid forms of these compounds with a stoichiometric amount of the appropriate base (such as Na, Ca, Mg, or K hydroxide, carbonate, bicarbonate, or the like).

A disintegrator, as used herein, refers to one or more of agar-agar, algins, calcium carbonate, carboxymethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone by dioxypropylcellulose, sodium starch glycolate, and starch, but is not limited thereto.

A binder, as used herein, refers to one or more of macrocrystalline cellulose, hydroxymethyl cellulose, and hydroxypropylcellulose, but is not limited thereto.

A filler, as used herein, refers to one or more of calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol, but is not limited thereto.

A lubricant, as used herein, refers to one or more of agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate, but is not limited thereto.

The composition according to the present invention may be administered to a patient by various routes. Examples of routes of administration include, but are not limited to, parenteral, e.g., intravenous, intradermal, subcutaneous, oral, intranasal (e.g., inhalation), transdermal (e.g., topical), transmucosal, and rectal administration. In an embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

In accordance with any of the embodiments, the composition according to the present invention can be administered orally to a subject in need thereof. Formulations suitable for oral administration can consist of (a) liquid solutions, such as an effective amount of the compound dissolved in diluents, such as water, saline, or orange juice and include an additive, such as cyclodextrin or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The composition according to the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the composition according to the present invention in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use.

The composition according to the present invention may be made into injectable formulations. The requirements for effective pharmaceutical carriers for injectable compositions are well known to those of ordinary, skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

The composition according to the present invention may be administered in an effective amount. An “effective amount” means an amount sufficient to show a meaningful benefit in a patient. Effective amounts may vary depending upon the biological effect desired in a patient, condition to be treated, and/or the specific characteristics of the composition according to the present invention and the individual. In this respect, any suitable dose of the composition can be administered to the patient (e.g., human), according to the biological effect desired or the type of disease to be treated. Various general considerations taken into account in determining the “effective amount” are known to those of skill in the art and are described, e.g., in Gilman et al, eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the composition according to the present invention desirably comprises about 0.1 mg per kilogram (kg) of the body weight of the patient (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

In an aspect, the dose of the composition according to the present invention desirably comprises about 0.1 millimole (mmol) per kilogram (kg) of the body weight of the patient (mmol/kg) to about 10 mmol/kg (e.g., about 0.1 mmol/kg, about 0.5 mmol/kg, about 1 mmol/kg, about 1.5 mmol/kg, about 2 mmol/kg/kg, about 2.5 mmol/kg/kg, about 3 mmol/kg, about 4 mmol/kg, about 5 mmol/kg, about 6 mmol/kg, about 7 mmol/kg, about 8 mmol/kg, about 9 mmol/kg, or about 10 mmol/kg).

Methods of diagnosing or monitoring include providing certain dosage amounts of an active agent to a patient. Dosage levels of each active agent of from about 0.1 millimole (mmol) per kilogram (kg) of the body weight of the patient (mmol/kg) to about 10 mmol/kg per day are useful in the methods of diagnosing or monitoring (e.g., about 0.1 mmol/kg, about 0.5 mmol/kg, about 1 mmol/kg, about 1.5 mmol/kg, about 2 mmol/kg/kg, about 2.5 mmol/kg/kg, about 3 mmol/kg, about 4 mmol/kg, about 5 mmol/kg, about 6 mmol/kg, about 7 mmol/kg, about 8 mmol/kg, about 9 mmol/kg, or about 10 mmol/kg).

Dosage levels of each active agent of from about 0, 1 mg to about 140 mg per kilogram of body weight per day are useful in the methods of diagnosing or monitoring (about 0.5 mg to about 7 g per patient per day). The amount, of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of the active agents are provided daily to a patient. Frequency of dosage may also vary depending on the compound used and the particular diagnosing or monitoring methods used. However, for many diagnosing or monitoring methods, a dosage regimen of 4 times daily or less can be used and in certain embodiments a dosage regimen of 1 or 2 times daily is used.

### Aspects of the Disclosure

Aspects, including embodiments, of the invention described herein may be beneficial alone or in combination, with one or more other aspects or embodiments. Without limiting the foregoing description, certain non-limiting aspects of the disclosure numbered 1-47 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A compound of Formula (I):

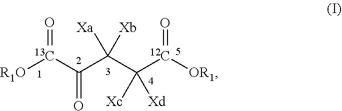

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

(2) The compound of aspect 1, wherein one of Xa and Xb is deuterium and the other is hydrogen and both Xc and Xd are hydrogen as shown in Formula (Ia):

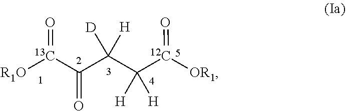

or a pharmaceutically acceptable salt thereof.

(3) The compound of aspect 1, wherein one of Xc and Xd is deuterium and the other is hydrogen and both Xa and Xb are hydrogen as shown in Formula (Ib):

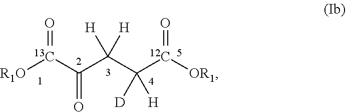

or a pharmaceutically acceptable salt thereof.

(4) The compound of aspect 1, wherein one of Xa and Xb is deuterium and the other is hydrogen and one of Xc and Xd is deuterium and the other is hydrogen as shown in Formula (Ic):

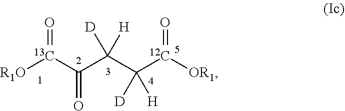

or a pharmaceutically acceptable salt thereof.

(5) The compound of aspect 1, wherein both Xa and Xb are deuterium and both Xc and Xd are hydrogen as shown in Formula (Id):

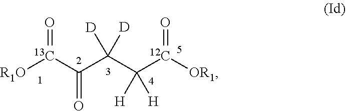

or a pharmaceutically acceptable salt thereof.

(6) The compound of aspect 1, wherein both Xa and Xb are hydrogen and both Xc and Xd are deuterium as shown in Formula (Ie):

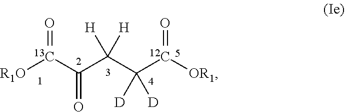

or a pharmaceutically acceptable salt thereof.

(7) The compound of aspect 1, wherein both Xa and Xb are deuterium and one of Xc and Xd is deuterium and the other is hydrogen as shown in Formula (If):

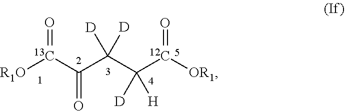

or a pharmaceutically acceptable salt thereof.

(8) The compound of aspect 1, wherein both Xc and Xd are deuterium and one of Xa and Xb is deuterium and the other is hydrogen as shown in Formula (Ig):

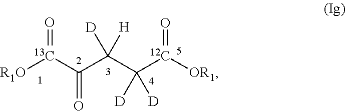

or a pharmaceutically acceptable salt thereof.

(9) The compound of aspect 1, wherein Xa, Xb, Xc, and Xd are deuterium as shown in Formula (Ih):

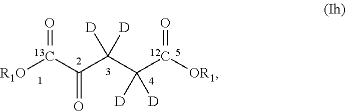

or a pharmaceutically acceptable salt thereof.

(10) The compound of any one of aspects 1-9, wherein each R₁ is independently selected from hydrogen, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl.

(11) The compound of any one of aspects 1-9, wherein each R₁ is independently selected from a C₁-C₆ alkyl.

(12) The compound of any one of aspects 1-9, wherein each R₁ is hydrogen.

(13) The compound of any one of aspects 1-9, wherein each R₁ is deuterium.

(14) The compound of any one of aspects 1-9, wherein each R₁ is a cation.

(15) A composition comprising an isotopic mixture of a compound of Formula (I):

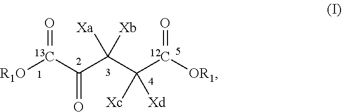

wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, and wherein at least 25% of a sum total deuterium and hydrogen atoms defined by Xa+Xb+Xc+Xd are deuterium,
or a pharmaceutically acceptable salt thereof.

(16) The composition of aspect 15, wherein at least 50% of a sum total deuterium and hydrogen atoms defined by Xa+Xb+Xc+Xd are deuterium.

(17) The composition of aspect 15, wherein at least 75% of a sum total deuterium and hydrogen atoms defined by Xa+Xb+Xc+Xd are deuterium.

(18) The composition of aspect 15, wherein at least 95% of a sum total deuterium and hydrogen atoms defined by Xa+Xb+Xc+Xd are deuterium.

(19) The composition of any one of aspects 15-18, wherein at least 50% of a sum total deuterium and hydrogen atoms defined by Xa+Xb are deuterium.

(20) The composition of any one of aspects 15-18, wherein at least 75% of a sum total deuterium and hydrogen atoms defined by Xa+Xb are deuterium.

(21) The composition of any one of aspects 15-18, wherein at least 95% of a sum total deuterium and hydrogen atoms defined by Xa+Xb are deuterium.

(22) The composition of any one of aspects 15-21, wherein at least 50% of a sum total deuterium and hydrogen atoms defined by Xc+Xd are deuterium.

(23) The composition of any one of aspects 15-21, wherein at least 75% of a sum total deuterium and hydrogen atoms defined by Xc+Xd are deuterium.

(24) The composition of any one of aspects 15-21, wherein at least 95% of a sum total deuterium and hydrogen atoms defined by Xc+Xd are deuterium.

(25) The composition of any one of aspects 15-24, wherein each R₁ is independently selected from hydrogen, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl.

(26) The composition of any one of aspects 15-24, wherein each R₁ is independently C₁-C₆ alkyl.

(27) The composition of any one of aspects 15-24, wherein each R₁ is hydrogen.

(28) The composition of any one of aspects 15-24, wherein each R₁ is deuterium.

(29) The composition of any one of aspects 15-24, wherein each R₁ is a cation.

(30) A pharmaceutical composition comprising a hyperpolarized compound of any one of aspects 1-14, or a pharmaceutically acceptable salt thereof, and a pharmaceutically acceptable carrier.

(31) A pharmaceutical composition comprising a hyperpolarized composition of any one of aspects 15-29, or a pharmaceutically acceptable salt thereof, and a pharmaceutically acceptable carrier.

(32) The pharmaceutical composition of aspect 30 or aspect 31 for use in a method of diagnosing or monitoring a patient having or suspected to have a cancer, the method comprising diagnosing or monitoring the patient by hyperpolarized ¹³C-MRI.

(33) The pharmaceutical composition for use according to aspect 32, wherein the method comprises identifying a mutation or mutations responsible for the cancer.

(34) The pharmaceutical composition for use according to aspect 33, wherein the method identifies an IDH1 mutation.

(35) A method of preparing a compound of aspect 1 or a pharmaceutically acceptable salt thereof, wherein each R₁ is independently selected from hydrogen, deuterium and a cation, the method comprising:
(i) reacting a compound of Formula (II) with deuterium oxide under basic conditions in an organic solvent and obtaining a compound of Formula (III):

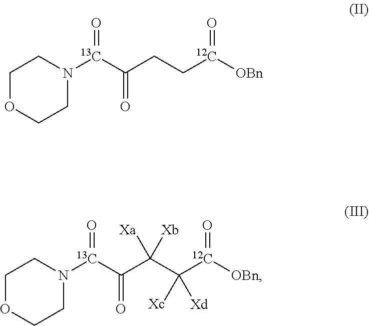

wherein Xa, Xb, c, and Xd are each independently hydrogen or deuterium, provided that at least one of Xa, Xb, Xc, and Xd is deuterium, and wherein Bn is benzyl; and
(ii) reacting the compound of Formula (III) with DCl and D₂O or HCl and H₂O to obtain the compound of aspect 1.

(36) A method of preparing an isotopic mixture of aspect 15, wherein each R₁ is independently selected from hydrogen, deuterium, and a cation, the method comprising:
(i) reacting a compound of Formula (II) with deuterium oxide under basic conditions in an organic solvent and obtaining an isotopic mixture of a compound of Formula (III):

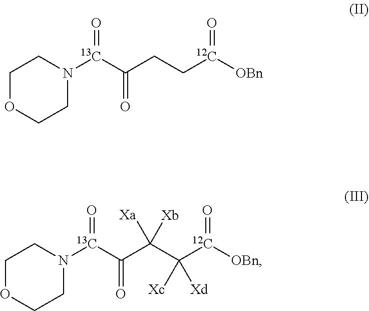

wherein Xa, Xb, Xc, and Xd are each independently hydrogen or deuterium, and wherein at least 25% of a sum total deuterium and hydrogen atoms defined by Xa+Xb+Xc+Xd are deuterium; and
(ii) reacting the isotopic mixture of the compound of Formula (III) with DCl and D₂O or HCl and H₂O to obtain the isotopic mixture of aspect 15.

(37) A method of preparing a hyperpolarized ketoglutarate compound comprising:
(i) providing a SABRE polarization transfer precatalyst;
(iii) combining the SABRE polarization transfer precatalyst with a ketoglutarate compound, parahydrogen, and optionally a co-ligand in a solvent to form a mixture comprising an active SABRE catalyst; and
(iv) hyperpolarizing the mixture comprising the active SABRE catalyst by exposing the mixture to a magnetic field or radiofrequency excitation to transfer the polarization from parahydrogen to the ketoglutarate compound to form the hyperpolarized ketoglutarate compound.

(38) The method of aspect 37, wherein the ketoglutarate compound is a compound of any one of aspects 1-14 or a pharmaceutically acceptable salt thereof or a composition of any one of aspects 15-32.

(39) The method of aspect 38, wherein each R₁ independently is a cation or C₁-C₆ alkyl.

(40) The method of any one of aspects 37-39, wherein the hyperpolarized ketoglutarate compound is of Formula (HP-I):

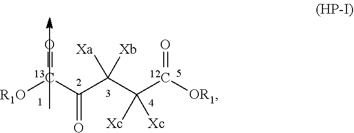

wherein the arrow in Formula (HP-I) designates the hyperpolarization,
wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

(41) The method of aspect 37, wherein the ketoglutarate compound is of Formula (IV):

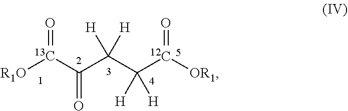

and wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl,
or a pharmaceutically acceptable salt thereof.

(42) The method of aspect 37 or 41, wherein the hyperpolarized ketoglutarate compound is of Formula (HP-IV):

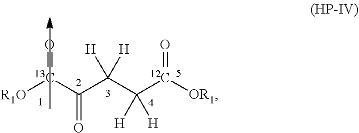

wherein the arrow in Formula (IP-IV) designates the hyperpolarization,
and wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl,
or a pharmaceutically acceptable salt thereof.

(43) The method of any one of aspects 37-42, wherein the SABRE polarization transfer precatalyst comprises a d-block element and one or more ligands.

(44) The method of aspect 43, wherein the one or more ligands are selected from phosphine ligand, carbene ligand, imidazole ligand, pincer chelating ligand, and a combination thereof.

(45) The method of any one of aspects 37-44, wherein the active SABRE catalyst-comprises a co-ligand.

(46) The method of aspect 45, wherein the co-ligand is a compound comprising a sulfoxide group.

(47) The method of aspect 46, wherein the compound comprising a sulfoxide group is selected from DMSO, phenyl methyl sulfoxide, phenyl chloromethyl sulfoxide, diphenyl sulfoxide, dibenzoyl sulfoxide, and dibutyl sulfoxide.

The following examples further illustrate the invention but, of course, should not be construed as in any way limiting its scope.

### Example 1

This example demonstrates a method of preparing 1-¹³C-3-D₂-4-D-5-¹²C ketoglutarate in accordance with an aspect of the invention:

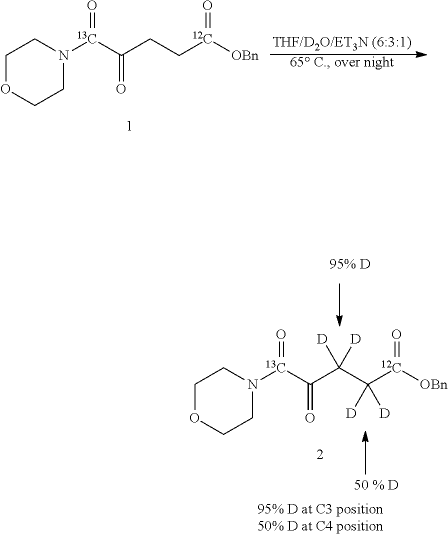 [SMILES: O=C(CC[12C](=O)OCc1ccccc1)[13C](=O)N1CCOCC1 ; [2H]C([2H])(C(=O)[13C](=O)N1CCOCC1)C([2H])([2H])[12C](=O)OCc1ccccc1]

In a 20 mL vial, compound 1 (1.3 grams, 4.2 mmol) was dissolved in THF/D₂O/Et₃N (6 mL: 3 mL: 1 mL). The vial was sealed and heated at 65° C. overnight (14 h). The reaction was cooled to room temperature and transferred to a 125 mL separating funnel and adding 10 mL D₂O. It was worked up with CH₂C₁₂ (2*50 mL), dried over anhydrous Na₂SO₄ and the filtrate was concentrated under reduced pressure to provide compound 2 (1.2 grams, 92% yield with 95% D at C-3 and 50% D at C-4). ¹H NMR (400 MHz, CdCl₃) δ 7.45-7.28 (m, 5H), 5.11 (s, 2H), 3.85-3.36 (m, 6H), 2.79-2.71 (m, 1H).

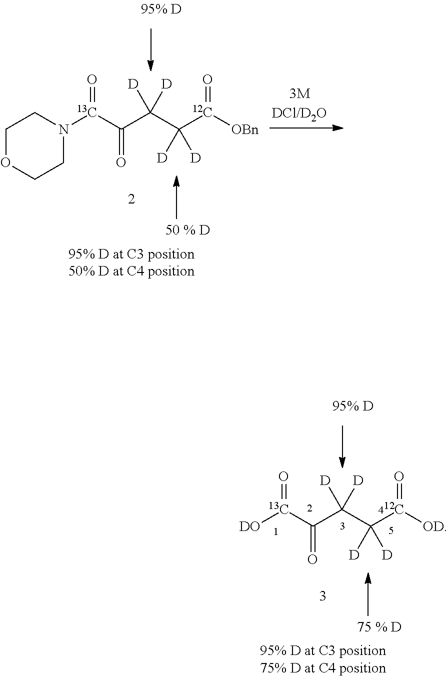

To a 3 molar solution of DCl in D₂O (50 mL), compound 2 (1.53 g, 5 mmole) was added. The reaction mixture was stirred at 80° C. for 16 h under the atmosphere of Argon. The solvent was removed in vacuo to yield a syrup. The resulting crude product was purified by ion chromatography on a formate resin (15 g, Bio-Rad AG 1-X8, 100-200 mesh). The column was conditioned with 4 M HCOOH and washed with D₂O until a pH of 6.5 was observed. The resulting crude product was dissolved in D₂O (10 mL) and loaded on to the column. The column was eluted using D₂O (200 mL) followed by 3 M DCl in D₂O (200 mL). The DCl phase was concentrated in vacuo and lyophilized from D₂O. A yellow-colored solid was obtained, which was crystallized from minimum amount of EtOAc at −20° C. to yield 3 (0.57 g, 75% yield). Any exposure of compound 3 to water leads to deuterium-hydrogen exchange of the carboxylic acid protons (i.e., R₁), and in some instances deuterium-hydrogen exchange at carbon positions 3 and 4. The deuterated compound was characterized by qualitative NMR using maleic acid as an internal standard. ¹³C NMR (101 MHz, Cd3CN) δ 169.13, 160.81. ¹H NMR of compound 2 in CDCl₃:

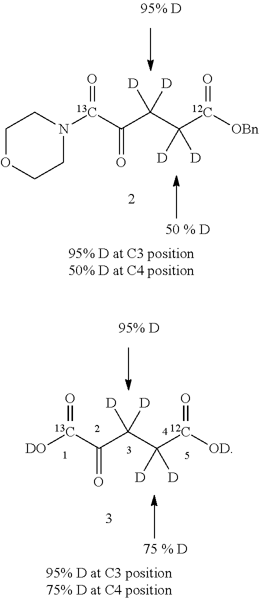

### Example 2

This example illustrates a method of synthesis of sodium 2-Oxopentanedioate-5-¹²C-1-¹³C in accordance with an aspect of the invention.

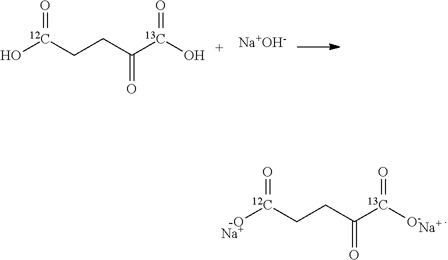

2-Oxopentanedioic-5-¹²C-1-¹³C acid (16 mg, 0.11 mmol) was dissolved in 200 μL of Deuterium oxide and sodium deuteroxide in 40% water (20 μL, 0.22 mmol) was added. The solution was lyophilized to give the desired product and further used as it was.

### Example 3

This example illustrates a method of synthesis of a SABRE catalyst in accordance with an aspect of the invention.

In a glovebox, a solution of 1,3-Bis(2,4,6-trimethylphenyl)-1,3-dihydro-2H-imidazol-2-ylidene (1 g, 3 mmol, Aldrich) in 20 mL THE was added dropwise (over 15 min) to a stirred solution of [{Ir(μ-Cl)(COD)}₂](1 g, 1 mmol, Aldrich) in 3 mL THF.

The solution was stirred for 12 h then removed from the glovebox and concentrated to about to ˜20% of the solution volume remained. Dried and deoxygenated pentane was added to allow precipitation of the complex. The yellow precipitate was filtered and recrystallized (1.07 g, 99% yield)

Ultra-high-purity hydrogen gas (Airgas) was fed into a Para-Hydrogen (pH₂) flow cryostat (Xeus technology LTD) and enriched to about 50% parahydrogen in the presence of a spin-exchange catalyst (Fe₂O₃) at liquid nitrogen temperature (77 K). The pH₂ flow was directed via PTFE tubing to a mass flow controller (MFC, Sierra Instruments SmartTrak 100 series) set at 90 ssc/m and directed to a conventional 5 mm NMR tube (Norell) to allow bubbling through the sample. The entire pH₂ line was pressurized to 96 psi.

Magnetic fields near or below ˜1 T were achieved with a home-built apparatus consisting of a solenoid coil placed inside a mu-metal shield (Magnetic Shield Corporation model No. ZG-206). At least once a day, the shield was degaussed using internal coils driven by a Variac. The solenoid has 41 mm diameter (40 mm core, 20 cm long windings with 220 turns AWG20 (0.9 mm) Cu wire and with 220Ω resistor in series. The solenoid coil was driven by commercial 1.5V batteries with a variable-resistance decade box in series to provide finer control of the internal magnetic field inside the shield. Typical values of the field within the shield were between ±1.2 T, with SABRE SHEATH experiments typically between 0.5 μT and 0.8 μT in the sample region. The values were monitored between SABRE experiments using a Lakeshore Cryotronics Gaussmeter (Model No. 475 DSP with HMMA-2512-VR Hall Probe.

NMR experiments were performed using a 1 T Magritek Spinsolve benchtop NMR spectrometer. All ¹³C NMR spectra were taken with ¹H decoupling turned off throughout the duration of the experiment. Time required to manually transfer the sample from the shield region to the magnet for low-field NMR acquisition was usually <5 s.

The efficient hyperpolarization transfer from p-H₂-derived hydrides to the ¹³C nuclear spin of [1-¹³C] ketoglutarate was possible by performing SABRE in sub-microtesla magnetic fields using SABRE in SHield Enables Alignment Transfer to Heteronuclei (SABRE-SHEATH) using a solution mixture of [IrCl(H)₂(DMSO)₂(IMes)], [1-¹³C] ketoglutarate) and p-H₂ in deuterated methanol.

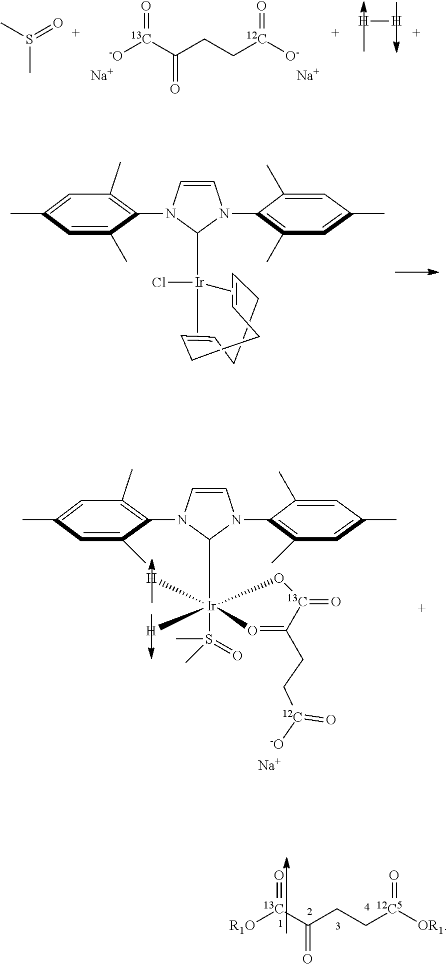

All experiments were performed with the solution containing 7.8 mM pre-catalyst, 45 mM DMSO and 10 mM [1-¹³C] Ketoglutarate in CD₃OD. The experiments were performed at room temperature, ˜100 sccm p-H₂ flow rate and 96 PSI p-H₂ overpressure. The parahydrogen used in this example came from a low-cost 50% p-H₂ generator.

For each experiment, the p-H₂ bubbling was applied for ˜1 min, the sample was quickly transferred to a 1.4 T NMR spectrometer for detection and the sample was then returned to the mu-metal shield to continue p-H₂ bubbling for the next experiment.

A schematic of the catalytic system for SABRE-SHEATH hyperpolarization is shown below. Activated Ir complex catalyst, [Ir(H₂)(η²-[1-¹³C] Ketoglutarate)(DMSO)(IMes)], transfers magnetization from p-H₂ to [1-¹³C] Ketoglutarate through a J-coupled spin network. Both p-H₂ and ketoglutarate have weak, transient binding to the iridium complex.

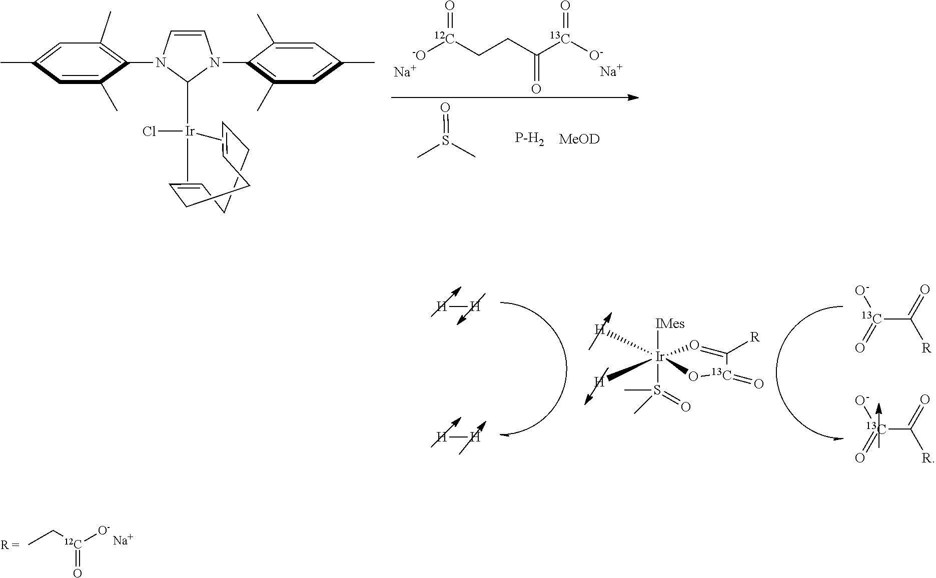

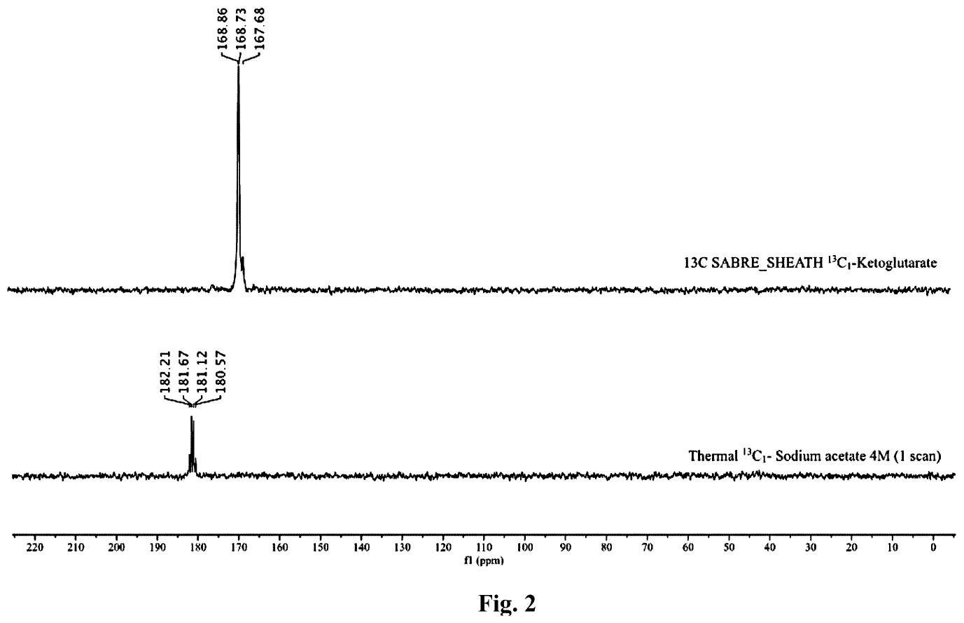

The result obtained from the SABRE-SHEATH experiment is shown in the FIG. 2. The top scan shows a single-scan HP ¹³C spectrum selected from SABRE-SHEATH experiments enhancement ε-3,000 and polarization is about P(¹³C)˜0.24%. Sample: 10 mM sodium [1-¹³C] Ketoglutarate, 45 mM DMSO, 7.8 mM Ir-IMes catalyst in methanol-d₄; spectrum acquired immediately following manual sample transfer to 1 T after 55 s p-H₂ bubbling at B_{T}=−0.2 μT.

The bottom scan in FIG. 2 depicts a single-scan thermally polarized ¹³C signal from 4 M sodium [1-¹³C]acetate using similar acquisition parameters.

### Example 4

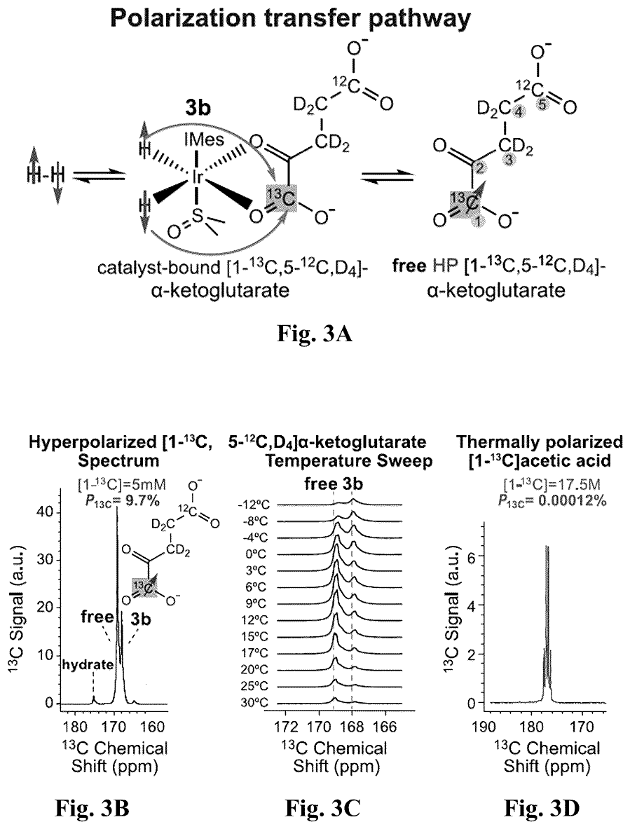

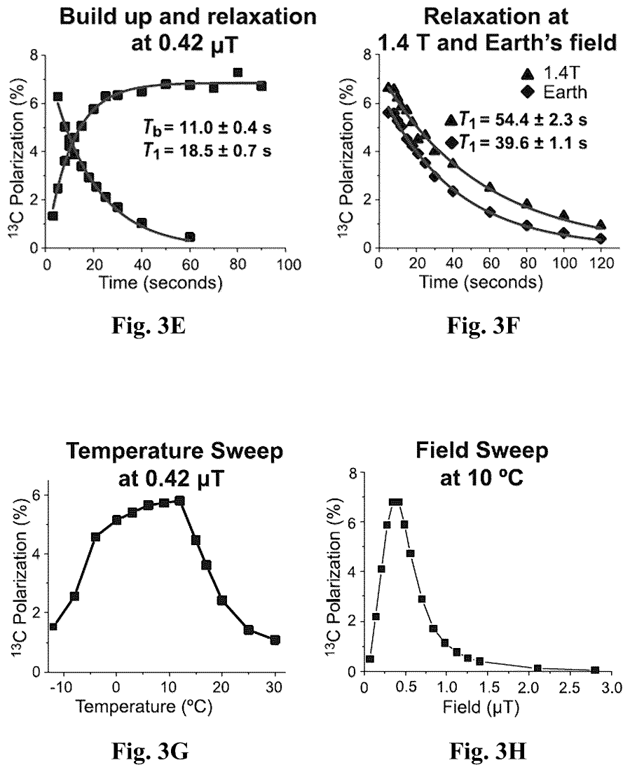

This example provides the ¹³C SABRE-SHEATH hyperpolarization results of the hyperpolarization of [1-¹³C, 5-¹²C; d₄] α-ketoglutarate, as prepared in Example 1, using a SABRE catalyst, as depicted in FIG. 3A.

A sample composition containing 5 mM [1-¹³C, 5-¹²C; d₄] α-ketoglutarate, 20 mM DMSO, 5 mM of the catalyst depicted in FIG. 3A, and 0.5 M water in CD₃OD was prepared. The sample was loaded in a medium walled NMR tube and argon was bubbled through the sample for 60 seconds to remove trapped air. The sample was next activated in the SABRE-SHEATH device, as described in Example 3, by bubbling parahydrogen (p-H₂) at a flow rate of 70 standard cubic centimeters per minute (sccm) and 100 PSI p-H₂ overpressure (total p-H₂ pressure ˜8 atm) for approximately 5 minutes or more. For each experimental run, p-H₂ was bubbled through the sample for 60 seconds and transferred to the 1.4 T benchtop SpinSolve NMR spectrometer for detection. The results are set forth in FIGS. 3B-3H. All experiments were performed at 1.4 T using a SpinSolve NMR spectrometer in CD₃OD at T_{T}=+10° C., unless noted otherwise.

FIG. 3B provides the ¹³C NMR spectrum of hyperpolarized [1-¹³C, 5-¹²C; d₄] α-ketoglutarate. FIG. 3C provides representative stacked variable-temperature ¹³C NMR spectra of 5 mM [1-¹³C, 5-¹²C; d₄] α-ketoglutarate, demonstrating the interplay between the hyperpolarized complex 3b in FIG. 3A and the ‘free’ peak as a function of temperature during the SABRE-SHEATH hyperpolarization process. FIG. 3D provides the corresponding ¹³C NMR spectrum of thermally polarized neat [1-¹³C]acetic acid, which was employed as a signal reference for computation of signal enhancements. FIG. 3E provides the buildup and decay of total ¹³C polarization of ¹³C-1 (i.e., integrating over all bound and free resonances) in [1-¹³C, 5-¹²C; d₄] α-ketoglutarate at B_{T}=0.42 μT. FIG. 3F provides the corresponding ¹³C-1 T₁ relaxation curves at the Earth's field and clinically relevant 1.4 T field of the benchtop spectrometer. FIG. 3G provides the total ¹³C polarization of ¹³C-1 [1-¹³C, 5-¹²C; d₄]α-ketoglutarate as a function of temperature. FIG. 3H provides the total ¹³C polarization of ¹³C-1 [1-¹³C, 5-¹²C; d₄] α-ketoglutarate as a function of magnetic transfer field.

In addition, the spin-lattice relaxation times were measured and were found to be as follows: At 3 T MRsolution MRI system in the solutions of DPBS with EDTA, T1 ([1-¹³C, 5-¹²C; d₄] α-ketoglutaric acid after flip angle correction)=97.1±0.416 seconds (apparent T1=39.1±0.414 seconds). For comparison to [1-¹³C, 5-¹²C; d₄] α-ketoglutaric acid, the spin-lattice relaxation times were measured and were found to be as follows: At 3 T MRsolution MRI system in the solutions of DPBS with EDTA T1 ([1-¹³C, 5-¹²C] protonated α-ketoglutaric, as prepared in Example 2, acid after flip angle correction)=63.0±0.835 seconds (apparent T1=32.1±0.824 seconds).

### Example 5

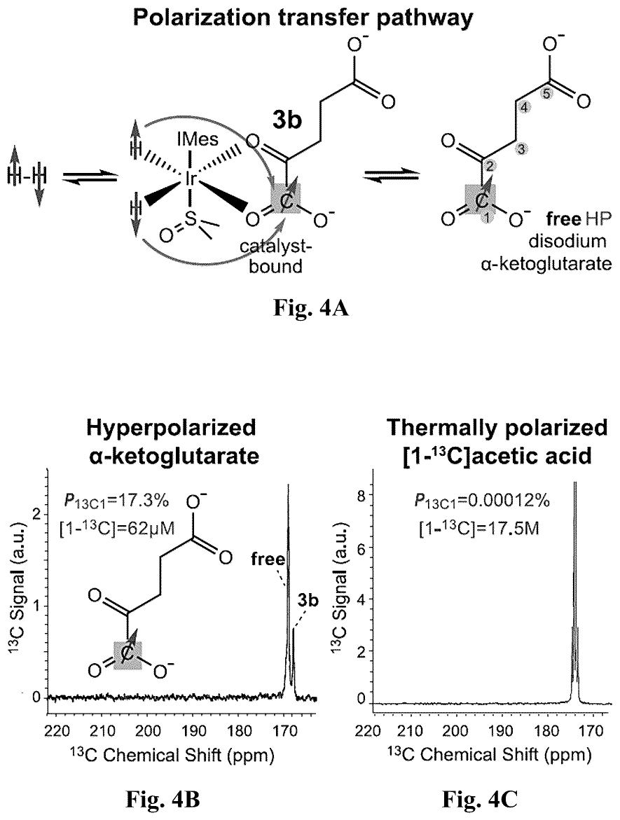

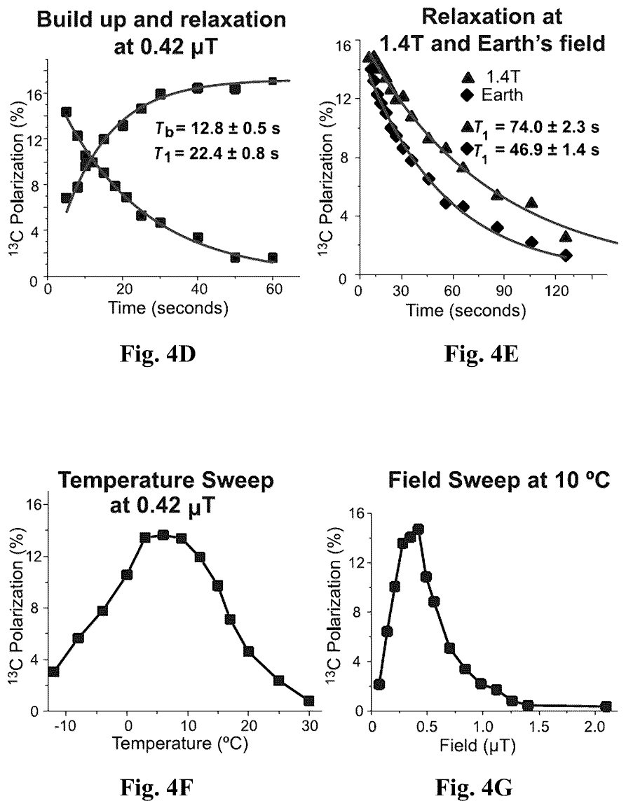

This example provides the ¹³C SABRE-SHEATH hyperpolarization results of the hyperpolarization of natural abundance disodium α-ketoglutarate, using a SABRE catalyst, as depicted in FIG. 4A.

A sample composition containing 5.6 mM natural abundance disodium α-ketoglutarate, 20 mM DMSO, 5 mM of the catalyst depicted in FIG. 4A, and 0.5 M water in CD₃OD was prepared. The sample was loaded in a medium walled NMR tube and argon was bubbled through the sample for 60 seconds to remove trapped air. The sample was next activated in the SABRE-SHEATH device, as described in Example 3, by bubbling parahydrogen (p-H₂) at a flow rate of 70 standard cubic centimeters per minute (sccm) and 100 PSI p-H₂ overpressure (total p-H₂ pressure ˜8 atm) for approximately 5 minutes or more. For each experimental run, p-H₂ was bubbled through the sample for 60 seconds and transferred to the 1.4 T benchtop SpinSolve NMR spectrometer for detection. The results are set forth in FIGS. 4B-4G. All experiments were performed at 1.4 T using a SpinSolve NMR spectrometer in CD₃OD at T_{T}=+10° C., unless noted otherwise.

FIG. 4B provides a representative ¹³C NMR spectrum of a 5.6 mM solution of natural-abundance disodium α-ketoglutarate obtained by performing SABRE-SHEATH at +10° C. in CD₃OD. FIG. 4C provides the corresponding ¹³C NMR spectrum of thermally polarized neat [1-¹³C]acetic acid, which was employed as a signal reference for computation of signal enhancements. FIG. 4D provides the buildup and decay of total ¹³C polarization (i.e., integrating over all bound and free resonances) in disodium α-ketoglutarate at B_{T}=0.42 μT and T_{T}=+10° C. FIG. 4E provides the total (bound+free)¹³C polarization decay of disodium α-ketoglutarate at the Earth's field and clinically relevant 1.4 T field of the benchtop spectrometer. FIG. 4F provides the total ¹³C polarization of ¹³C-1 in natural abundance disodium α-ketoglutarate as a function of temperature. FIG. 4G provides the total ¹³C polarization of ¹³C-1 in natural abundance disodium α-ketoglutarate as a function of magnetic transfer field.

The results from Examples 4 and 5 show that (i)¹³C labelling at position 1 of α-ketoglutarate enables spectroscopic sensing, (ii) deuteration of position 3 and position 4 maximizes the lifetime of the ¹³C-1 hyperpolarized state in aqueous media at 3%, and (iii) ¹²C labelling at position 5 of α-ketoglutarate minimizes the background signal from this resonance.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of Formula (I): 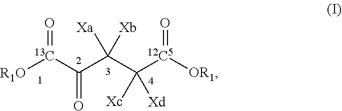
wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

2.-9. (canceled)

10. The compound of claim 1, wherein each R₁ is independently selected from hydrogen, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl.

11. (canceled)

12. (canceled)

13. The compound of claim 1, wherein each R₁ is deuterium.

14. The compound of claim 1, wherein each R₁ is a cation.

15. A composition comprising an isotopic mixture of a compound of Formula (I): 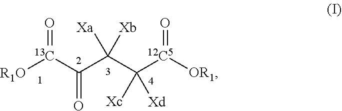
wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, and wherein at least 25% of a sum total deuterium and hydrogen atoms defined by Xa+Xb+Xc+Xd are deuterium,
or a pharmaceutically acceptable salt thereof.

16. The composition of claim 15, wherein at least 50% of a sum total deuterium and hydrogen atoms defined by Xa+Xb+Xc+Xd are deuterium.

17. (canceled)

18. (canceled)

19. The composition of claim 15, wherein at least 50% of a sum total deuterium and hydrogen atoms defined by Xa+Xb are deuterium.

20. (canceled)

21. (canceled)

22. The composition of claim 15, wherein at least 50% of a sum total deuterium and hydrogen atoms defined by Xc+Xd are deuterium.

23. (canceled)

24. (canceled)

25. The composition of claim 15, wherein each R₁ is independently selected from hydrogen, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl.

26. (canceled)

27. (canceled)

28. The composition of claim 15, wherein each R₁ is deuterium.

29. The composition of claim 15, wherein each R₁ is a cation.

30. A pharmaceutical composition comprising a hyperpolarized compound of claim 1, or a pharmaceutically acceptable salt thereof, and a pharmaceutically acceptable carrier.

31. A pharmaceutical composition comprising a hyperpolarized composition of claim 15, or a pharmaceutically acceptable salt thereof, and a pharmaceutically acceptable carrier.

32. The pharmaceutical composition of claim 30 for use in a method of diagnosing or monitoring a patient having or suspected to have a cancer, the method comprising diagnosing or monitoring the patient by hyperpolarized ¹³C-MRI.

33. (canceled)

34. (canceled)

35. A method of preparing a compound of claim 1 or a pharmaceutically acceptable salt thereof, wherein each R₁ is independently selected from hydrogen, deuterium and a cation, the method comprising:
(i) reacting a compound of Formula (II) with deuterium oxide under basic conditions in an organic solvent and obtaining a compound of Formula (III): 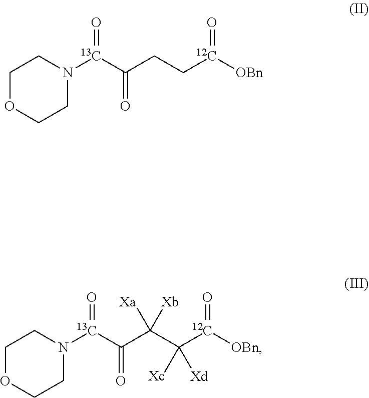
wherein Xa, Xb, Xc, and Xd are each independently hydrogen or deuterium, provided that at least one of Xa, Xb, Xc, and Xd is deuterium, and wherein Bn is benzyl; and
(ii) reacting the compound of Formula (III) with DCl and D₂O or HCl and H₂O to obtain the compound of claim 1.

36. A method of preparing an isotopic mixture of claim 15, wherein each R₁ is independently selected from hydrogen, deuterium, and a cation, the method comprising:
(i) reacting a compound of Formula (II) with deuterium oxide under basic conditions in an organic solvent and obtaining an isotopic mixture of a compound of Formula (III): 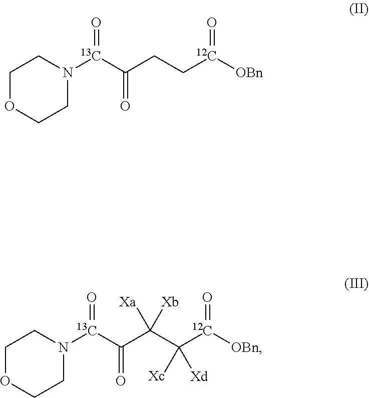
wherein Xa, Xb, Xc, and Xd are each independently hydrogen or deuterium, and wherein at least 25% of a sum total deuterium and hydrogen atoms defined by Xa+Xb+Xc+Xd are deuterium; and
(ii) reacting the isotopic mixture of the compound of Formula (III) with DCl and D₂O or HCl and H₂O to obtain the isotopic mixture of claim 15.

37. A method of preparing a hyperpolarized ketoglutarate compound comprising:
(i) providing a SABRE polarization transfer precatalyst;
(ii) combining the SABRE polarization transfer precatalyst with a ketoglutarate compound, parahydrogen, and optionally a co-ligand in a solvent to form a mixture comprising an active SABRE catalyst; and
(iii) hyperpolarizing the mixture comprising the active SABRE catalyst by exposing the mixture to a magnetic field or radiofrequency excitation to transfer the polarization from parahydrogen to the ketoglutarate compound to form the hyperpolarized ketoglutarate compound.

38. The method of claim 37, wherein the ketoglutarate compound is a compound of Formula (I): 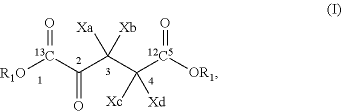
wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

39. (canceled)

40. The method of claim 37, wherein the hyperpolarized ketoglutarate compound is of Formula (HP-I): 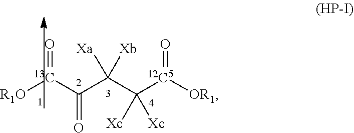
wherein the arrow in Formula (HP-I) designates the hyperpolarization,
wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl; and
wherein Xa, Xb, Xc, and Xd are each independently hydrogen or deuterium, provided that at least one of Xa, Xb, Xc, and Xd is deuterium,
or a pharmaceutically acceptable salt thereof.

41. The method of claim 37, wherein the ketoglutarate compound is of Formula (IV): 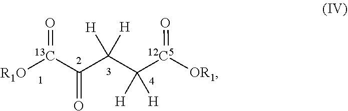
and wherein each R₁ is independently selected from hydrogen, deuterium, a cation, C₁-C₆ alkyl, C₃-C₇ cycloalkyl, (C₃-C₇ cycloalkyl)C₁-C₆ alkyl, (heterocycloalkyl)C₁-C₆ alkyl, (heteroaryl)C₁-C₆ alkyl, and (aryl)C₁-C₆ alkyl,
or a pharmaceutically acceptable salt thereof.

42.-47. (canceled)
